(19) United States
(12) Patent Application Publication
(10) Pub. No.: US 2016/0354373 A1
(43) Pub. Date: Dec. 8, 2016

(54) METHODS OF TREATING ACUTE T CELL LEUKEMIA

(71) Applicants: Iannis AIFANTIS, Brooklyn, NY (US); Anastasia TIKHONOVA, New York, NY (US)

(72) Inventors: Iannis AIFANTIS, Brooklyn, NY (US); Anastasia TIKHONOVA, New York, NY (US)

(73) Assignee: New York University, New York, NY (US)

(21) Appl. No.: 15/174,449

(22) Filed: Jun. 6, 2016

Related U.S. Application Data
(60) Provisional application No. 62/171,797, filed on Jun. 5, 2015.

(51) Int. Cl. A61K 31/506; A61K 31/4709; A61K 31/4427; A61K 45/06; A61K 31/395

(52) U.S. Cl. CPC A61K 31/506; A61K 45/06; A61K 31/395; A61K 31/4427; A61K 31/4709

(57) ABSTRACT

The present invention is directed to methods of treating T-cell acute lymphoblastic leukemia. This method involves selecting a subject having T-cell acute lymphoblastic leukemia and administering, to the selected subject, a therapeutic agent that inhibits CXCR4-CXCL12 signaling at a dosage effective to treat the T-cell acute lymphoblastic leukemia in the subject. A method of inhibiting T-cell acute lymphoblastic leukemia cell proliferation and/or survival is also disclosed.

This application claims the benefit of U.S. Provisional Patent Application Ser. No. 62/171,797, filed Jun. 5, 2015, which is hereby incorporated by reference in its entirety.

### FIELD OF THE INVENTION

The present invention is directed to methods of treating and preventing acute T cell leukemia.

### BACKGROUND OF THE INVENTION

Acute lymphoblastic leukemia (ALL) is the most common of childhood cancers, and 15-20% of ALL cases are T lineage (T-ALL) (Pui et al., “Challenging Issues in Pediatric Oncology,” Nat. Rev. Clin. Oncol. 8:540-549 (2011)). A quarter of childhood T-ALL patients relapse within 5 years of treatment and receive a dismal prognosis (Nguyen et al., “Factors Influencing Survival After Relapse From Acute Lymphoblastic Leukemia: a Children's Oncology Group Study,” Leukemia 22:2142-2150 (2008)). Factors predicting poor survival of relapsed childhood ALL patients include T lineage disease (Ma et al., “Early T-cell Precursor Leukemia: a Subtype of High Risk Childhood Acute Lymphoblastic Leukemia,” Frontiers in Medicine 6:416-420 (2012)) and isolated bone marrow involvement, both of which have a less than 25% five year survival rate (Bhojwani and Pui, “Relapsed Childhood Acute Lymphoblastic Leukaemia,” The Lancet Oncology 14:e205-217 (2013); Nguyen et al., “Factors Influencing Survival After Relapse From Acute Lymphoblastic Leukemia: a Children's Oncology Group Study,” Leukemia 22:2142-2150 (2008)). Therefore, the search for more effective, less toxic treatments continues.

A series of seminal papers has demonstrated that the majority of T-ALL cases are driven by activating NOTCH1 mutations and activation of downstream pathways, including MYC signaling, which has been shown to be essential for T-ALL cell proliferation and leukemia-initiating cell (LIC) activity (Girard et al., “Frequent Provirus Insertional Mutagenesis of Notch1 in Thymomas of MMTVD/myc Transgenic Mice Suggests a Collaboration of c-myc and Notch1 for Oncogenesis,” Genes & Dev. 10:1930-1944 (1996); King et al., “The Ubiquitin Ligase FBXW7 Modulates Leukemia-initiating Cell Activity by Regulating MYC Stability,” Cell 153:1552-1566 (2013); Pear et al., “Exclusive Development of T Cell Neoplasms in Mice Transplanted With Bone Marrow Expressing Activated Notch Alleles,” J. Exp. Med. 183:2283-2291 (1996); Weng et al., “Activating Mutations of NOTCH1 in Human T Cell Acute Lymphoblastic Leukemia,” Science 306:269-271 (2004)).

Increasing evidence suggests that leukemic stem cells actively engage in crosstalk with the bone marrow microenvironment to regulate their proliferation and survival (Ayala et al., “Contribution of Bone Microenvironment to Leukemogenesis and Leukemia Progression,” Leukemia 23:2233-2241 (2009)). Similarities between leukemia-initiating cells (LIC) and hematopoietic stem cells (HSC) have raised the hypothesis that LIC require a specialized microenvironment to survive, and that disrupting this niche may be a promising therapeutic strategy (Scadden, “Nice neighborhood: Emerging Concepts of the Stem Cell Niche,” Cell 157:41-50 (2014)). During the last decade, cellular components of the HSC niche have been identified and analyzed (Morrison and Scadden, “The Bone Marrow Niche for Haematopoietic Stem Cells,” Nature 505:327-334 (2014)). Imaging studies showed that HSCs tended to localize in the proximity of blood vessels, focusing the field's attention on the perivascular niche. In vivo depletion of Nestin⁺ CXCL12^{high} mesenchymal stem cells (MSCs) that surround blood vessels resulted in impaired progenitor cell homing (Mendez-Ferrer et al., “Mesenchymal and Haematopoietic Stem Cells Form a Unique Bone Marrow Niche,” Nature 466:829-834 (2010)). Elegant work by Ding et al. and Greenbaum et al. identified endothelial and perivascular populations as distinct and specialized niches supporting HSC homeostasis (Ding and Morrison, “Haematopoietic Stem Cells and Early Lymphoid Progenitors Occupy Distinct Bone Marrow Niches,” Nature 495:231-235 (2013); Greenbaum et al., “CXCL12 in Early Mesenchymal Progenitors is Required for Haematopoietic Stem-Cell Maintenance,” Nature 495:227-230 (2013)).

The present invention is directed to overcoming these and other deficiencies in the art.

### SUMMARY OF THE INVENTION

A first aspect of the present invention is directed to a method of treating T-cell acute lymphoblastic leukemia in a subject. This method involves selecting a subject having T-cell acute lymphoblastic leukemia and administering, to the selected subject, a therapeutic agent that inhibits CXCR4-CXCL12 signaling at a dosage effective to treat the T-cell acute lymphoblastic leukemia in the subject.

Another aspect of the present invention is directed to a method of inhibiting T-cell acute lymphoblastic leukemia cell proliferation and/or survival. This method involves administering to a population of T-cell acute lymphoblastic leukemia cells an inhibitor of CXCR4-CXCL12 signaling at a dosage effective to inhibit the T-cell acute lymphoblastic leukemia cells proliferation and/or survival.

In the study described herein, the role of CXCL12 in T-ALL pathogenesis was examined, and the mechanisms underlying interaction of leukemia with its microenvironment were explored. It was shown that although both T-ALL LIC and HSC interact with CXCL12-expressing stromal cells, they depend on distinct niche subpopulations and manifest a diametrically distinct response to CXCR4 antagonism. The results map for the first time the T-ALL bone marrow microenvironment and introduce a novel therapeutic avenue for this aggressive blood malignancy.

### BRIEF DESCRIPTION OF THE DRAWINGS

FIGS. 1A-D demonstrate that T-ALL cells do not produce CXCL12 in a cell-autonomous manner. FIG. 1A shows the abundance of Cxcl12 mRNA expressed relative to Actb mRNA in purified bone marrow DsRed+ cells from Cxcl12-DsRed mice (2 samples) and T-ALL cells (2 samples) measured by RT-qPCR. Error bars represent +/−SD. FIG. 1B shows the experimental design on the left, and representative FACS analysis of splenocytes from hosts reconstituted with Notch1-ΔE(N1-ΔE)-GFP⁺ cells generated from c-kit⁺ Cxcl12-DsRed cells or wildtype littermate progenitor cells on the right. FIG. 1C is a representative histogram of Cxcl12-DsRed expression in N1-ΔE-GFP⁺ cells from spleen, lymph nodes, and bone marrow of leukemic mice. CD45-DsRed+ cells from bone marrow of Cxcl12-DsRed mice were used as positive control for dDsRed expression. FIG. 1D is a genomic PCR from Cxcl12-DsRed, WT-N1-ΔE-GFP⁺, and Cxcl12-DsRed-N1-ΔE-GFP⁺ cells from spleen, lymph nodes, and bone marrow confirming genotype of Cxcl12-DsRed and WT T-ALL leukemic cells. Data from FIGS. 1B-D are representative of two independent experiments (n=6).

FIGS. 2A-I demonstrate that T-ALL cells interact with a CXCL12-producing niche in the bone marrow. FIGS. 2A-C show three different representative immunofluorescence stainings of femur sections from CXCL12-DsRed mice transplanted with GFP⁺ T-ALL cells (n=4). FIGS. 2D-E show two different representative high-resolution two photon images from CXCL12-DsRed mice transplanted with GFP⁺ T-ALL cells. FIG. 2F shows a representative high-resolution two-photon image from CXCL12-DsRed mice transplanted with GFP⁺ T-ALL and CFP⁺ CD4⁺ T cells. FIG. 2G shows the percentage of GFP⁺ T-ALL cells in contact with Cxcl12-Dsred⁺ cells. Each data point is taken from a different image. FIGS. 2H-I show track velocity (FIG. 2H) and displacement velocity (FIG. 2I) of GFP⁺ T-ALL cells and CFP⁺ CD4⁺ T cells. Error bars represent +/−SD. Unless otherwise stated, each panel reflects data from at least three independent experiments.

FIGS. 3A-G demonstrate that CXCL12 production by vascular endothelial cells maintains T-ALL. FIG. 3A is a schematic representation of CXCL12 producing populations in bone marrow. FIG. 3B shows two photon images of bone marrow from Cxcl12-DsRed, VEcad-cre;LoxP-tdTomato, Lepr-cre;LoxP-tdTomato, and Col2.3-cre;LoxPtdTomato animals 1 week after transfer of GFP⁺ T-ALL cells. FIG. 3C shows the frequency of co-localization between GFP⁺ T-ALL and Dsred/tdTomato niche cells from Cxcl12-Dsred, VEcad-cre;LoxP-tdTomato, Lepr-cre;LoxP-tdTomato, and Col2.3-cre;LoxP-tdTomato animals 1 week after transfer of leukemic cells. At least three animals were used for each condition. Error bars represent +/−SD. FIG. 3D shows the representative frequency of T-ALL GFP⁺ cells in blood 11 days and 19 days after secondary transplantation into VEcad-cre;Cxcl12^{fl/fl}, Lepr-cre;Cxcl12^{fl/fl}, or control hosts. FIG. 3E shows the absolute numbers of T-ALL cells in lymph nodes, spleen, and bone marrow 25 days post secondary transplantation of GFP⁺ T-ALL cells into VEcad-cre;Cxcl12^{fl/fl}, Lepr-cre;Cxcl12^{fl/fl}, or control hosts. Bone marrow numbers represent cells harvested from tibias and femurs. Data is representative of three experiments for VEcad-cre;Cxcl12^{fl/fl} (n=6) or littermate sex-matched control animals (n=7) and two experiments for Lepr-cre;Cxcl12^{fl/fl} (n=9) or control hosts (n=8). Error bars represent +/−SD. FIG. 3F is an image of representative spleens from VEcad-cre;Cxcl12^{fl/fl} or control animals. FIG. 3G shows histology of lungs and liver from VEcad-cre;Cxcl12^{fl/fl} or control animals.

FIGS. 4A-F show the visualization of bone marrow niches. FIG. 4A shows immunofluorescence images of bone marrow sections from: (a-b) Cxcl12DsRed/+ mice; (c-d) Col2.3-cre;LoxPtdTomato mice, in which tdTomato is expressed in osteoblasts, marking the endosteal niche of bone marrow; (e-f) Lepr-cre;LoxP-tdTomato animals, with perivascular cells labeled with tdTomato; (g-h) VEcad-cre;LoxP-tdTomato animals, in which tdTomato labels the vascular niche. Nuclei were stained with 4′,6-diamidino-2-phenylindole (DAPI, blue). Images are representative of 3 different mice of each genotype. FIG. 4B shows two photon images of bone marrow from CXCL12-DsRed, VEcad-cre;LoxP-tdTomato, Lepr-cre;LoxP-tdTomato and Col2.3-cre;LoxP-tdTomato animals 1 week after transfer of GFP⁺ ETP T-ALL or ENU-induced T-ALL (NOTCH1 PEST G7084 (ins)C7085) cells. FIG. 4C shows the frequency of co-localization between GFP⁺ ETP T-ALL or PEST-mutant T-ALL and DsRed/tdTomato⁺ niche cells from CXCL12-DsRed, VEcad-cre;LoxP-tdTomato, and Col2.3-cre;LoxP-tdTomato animals 1 week after transfer of leukemic cells. At least 3 animals were used for each host genotype. Error bars represent +/−SD. FIG. 4D shows representative FACS analysis showing the frequency of Notch1-ΔE-GFP⁺ T-ALL cells in the thymus of control (n=7), VEcad-cre;Cxcl12^{fl/fl} (n=6), and Lepr-cre;Cxcl12^{fl/fl} (n=4) animals 4-6 weeks after T-ALL cell transfer. FIG. 4E shows the absolute T-ALL cell numbers in the thymus 4-6 weeks after transplantation of GFP⁺ Notch1-ΔE-GFP⁺ T-ALL cells into VEcad-cre;Cxcl12^{fl/fl}, Lepr-cre;Cxcl12^{fl/fl}, or control hosts. Error bars represent +/−SD. Data for FIGS. 4D-E was pooled from 2 independent experiments. FIG. 4F shows representative immunofluorescence staining of thymus from control, VEcad-cre;Cxcl12^{fl/fl}, and Lepr-cre;Cxcl12^{fl/fl} animals after establishment of Notch1-ΔE-GFP⁺ T-ALL. At least 2 animals were assessed for each host genotype.

FIGS. 5A-E demonstrate that CXCR4 is highly expressed on the surface of mouse and human T-ALL cells. FIG. 5A shows surface CXCR4 expression on T-ALL cells from a representative leukemic mouse and normal T cells from a healthy control. T-ALL cells were identified as CD4⁺CD8⁺GFP⁺. FIG. 5B shows surface CXCR4 mean fluorescence intensity (MFI) on T-ALL cells and normal T cells from the indicated organs. The graph pools data from 3-5 pairs of leukemic mice and controls. Bars represent mean MFI. FIG. 5C shows the abundance of Cxcr4 mRNA expressed relative to Hprt mRNA in purified normal CD4⁺CD8⁺ thymocytes, normal spleen CD4⁺ T cells, and CD4⁺CD8⁺ T-ALL cells, measured by RT-qPCR. Bars represent the mean. FIG. 5D shows the surface CXCR4 expression on human peripheral blood CD4⁺CD3⁺ and CD8⁺CD3⁺ lymphocytes from healthy controls. FIG. 5E shows the surface CXCR4 expression on primary bone marrow human biopsies from T-ALL patients expanded in immunodeficient hosts (gated on hCD45) (patients 1-4 and 8; patient 4 had ETP T-ALL) and primary T-ALL bone marrow biopsies (patients 5-7).

FIGS. 6A-B shows CXCR7 and CXCR4 expression in T-ALL. FIG. 6A shows T-ALL cells from mouse spleen and the CUTLL1 human T-ALL cell line that were stained for surface CXCR7 or fixed and permeabilized for analysis of intracellular CXCR7. Data are representative of T-ALL from 3 different mice or staining of CUTLL1 cells on 3 separate days. FIG. 6B shows processed T-ALL patient microarray expression data downloaded from (Zhang et al., “The Genetic Basis of Early T-cell Precursor Acute Lymphoblastic Leukaemia,” Nature 481:157-163 (2012), which is hereby incorporated by reference in its entirety). All of the data was acquired on GPL96 Affymetrix Human Genome U133A Array. The data represents T cells from 24 patients and T-ALL samples from 83 patients. Data was first converted to logarithmic scale when necessary, and then quantile normalized across samples. Pairwise t-test per gene probe was used to determine significant differences between sample categories (T-ALL and physiological T cells). For each boxplot, the middle line inside the box corresponds to the median. The upper and lower horizontal lines correspond to the first and third quartiles (the 25th and 75th percentiles). The upper whisker extends from top horizontal line to the highest value that is within 1.5×IQR of that line, where IQR is the inter-quartile range, or distance between the first and third quartiles. Similarly, the lower whisker extends from the bottom horizontal line to the lowest value within 1.5×IQR of that line.

FIGS. 7A-C demonstrate that T-ALL induction and CXCR4 deletion using Cxcr4^{f/f} Mx1-Cre+ or littermate control bone marrow progenitor cells. FIG. 8A is representative FACS analysis shows the frequency of Notch1-ΔE-GFP⁺ cells, and expression of CD4, CD8 and surface CXCR4 on GFP⁺ cells, in peripheral blood one day before poly(I:C) treatment (day 0). Data represents 2 experiments (n=7-8). FIG. 8B shows the decreased abundance of Cxcr4 mRNA relative to Hprt mRNA in Cxcr4^{f/f} Mx1-Cre⁺ T-ALL cells compared to Cxcr4^{f/f} T-ALL cells 10 days after administration of poly(I:C), measured by RT-qPCR. Bars represent the mean. FIG. 8C shows the frequency and number of GFP⁺ T-ALL cells, and number of GFP⁺ CD4⁺CD8⁺ blasts, remaining in spleen and bone marrow of mice shown in FIG. 8E with Cxcr4-deficient T-ALL 215 days post-poly(I:C) treatment. Bars represent the mean (n=10).

FIGS. 8A-E demonstrate that deletion of Cxcr4 reduces T-ALL burden and significantly prolongs survival. FIG. 9A is a schematic representation of the experimental design. FIG. 9B shows representative surface CXCR4 staining on Cxcr4^{f/f} Mx1-Cre⁺ or littermate control (Cxcr4^{+/+}Mx1-Cre⁺ or Cxcr4^{f/f}) GFP⁺ T-ALL cells in the spleen one month after they were treated with poly(I:C). FIG. 9C shows the number of GFP⁺ Cxcr4^{f/f} Mx1-Cre⁺ or littermate control (Cxcr4^{+/+}Mx1-Cre⁺ or Cxcr4^{f/f}) T-ALL cells in the indicated tissues 1 month after treatment with poly(I:C) (initiated after GFP⁺ cells represented >10% of peripheral blood lymphocytes). Bars represent the mean. Data are pooled from 2 experiments (n=7-8). FIG. 9D shows representative immunofluorescence staining of a femur section from a mouse that received Cxcr4^{f/f} Mx1-Cre⁺ or littermate control T-ALL, 1 month after poly(I:C) treatment. FIG. 9E is a Kaplan-Meier survival graph of mice with Cxcr4^{f/f} Mx1-Cre⁺ (n=10) or littermate control (Cxcr4^{+/+} Mx1-Cre⁺, n=1) T-ALL following poly(I:C) treatment (initiated after T-ALL cells reached ˜10% of blood lymphocytes; first poly(I:C) injection was defined as day 1).

FIGS. 9A-E demonstrate the effects of CXCR4 depletion on leukemic cell localization and survival. FIG. 10A is a schematic representation of experimental design. FIG. 10B shows the frequency of transplanted+Cxcr4^{f/f} Mx1-Cre⁺ or littermate control T-ALL GFP⁺ cells in the blood prior to poly(I:C) treatment on the left. The frequency of GFP⁺ leukemic cells and levels of CXCR4 in the bone marrow 48 hours after the second dose of poly(I:C) is shown on the right. FIG. 10C shows representative immunofluorescence staining of a femur section from CXCL12-dsRed hosts transplanted with GFP⁺ Cxcr4^{f/f} Mx1-Cre⁺ or littermate control T-ALL cells 48 hours after poly(I:C) treatment. FIG. 10D shows the experimental design (top) and representative high-resolution two-photon image from VEcad-cre;LoxP-tdTomato mice transplanted with Cxcr4^{f/f} Mx1-Cre⁺ or littermate control T-ALL 24 hours after poly(I:C) treatment (bottom). Arrows indicate presence or absence of leukemic cells on top of the vessels. FIG. 10E shows Annexin V staining on GFP⁺ Cxcr4^{f/f} Mx1-Cre⁺ or littermate control T-ALL cells in blood and bone marrow 24 hours after poly(I:C) treatment (n=3).

FIGS. 10A-B shows loss of Cxcl12 in vascular endothelial cells induces apoptosis in T-ALL cells. FIG. 10A shows representative Annexin V staining on GFP⁺ T-ALL cells from spleens of control or VEcad-cre;Cxcl12^{fl/fl} hosts 6 weeks post-transplantation. FIG. 10B shows the frequency of Annexin V⁺ cells in spleens of control or VEcad-cre;Cxcl12^{fl/fl} in hosts (control=3 mice; VEcad-cre;Cxcl12^{fl/fl}=4 mice). Error bars represent +/−SD.

FIGS. 11A-F demonstrate that small molecule CXCR4 antagonists efficiently suppress growth of murine and human T-ALL. FIG. 11A shows T-ALL was induced by transfer of Notch1-ΔE transduced progenitors. After GFP⁺ leukemic cells reached 5% of white blood cells, osmotic pumps filled with AMD3465 (20 nmol/hour) or vehicle (PBS) were implanted subcutaneously (s.c.). The graphs show T-ALL cell numbers in the indicated tissues 2 weeks after treatment initiation. Bars represent the mean. FIGS. 11B-F show NOD-SCID mice injected with leukemia cells from patient 1 or 2 (represented in FIG. 5E). After leukemia reached 5% of white blood cells, osmotic pumps filled with AMD3465 (25 nmol/hour) or vehicle were implanted s.c. Analysis was 2 weeks after treatment initiation. FIG. 11B shows a representative flow cytometric analysis of hCD45 expression amongst peripheral blood lymphocytes. Values represent the mean frequency of hCD45⁺ cells+/−SEM. FIGS. 11C-D shows the number of hCD45⁺ leukemia cells per mL of peripheral blood (FIG. 11C) or in the spleen (FIG. 11D) of animals treated with AMD3465 or vehicle. Bars represent the mean. FIG. 11E is an image of representative spleens and lymph nodes (patient 1 xenograft). FIG. 11F shows histology of lungs, liver and brain (patient #1 xenograft). Data for FIGS. 11B-F are representative of 6-7 mice per group using cells derived from patient 1, or 5-6 mice per group using cells derived from patient 2.

FIG. 12 demonstrates CXCR4 antagonism of primary human leukemia xenografts. An image of representative spleens and bone marrow from NOD/SCID mice xenografted with primary human T-ALL cells (patient #2), after 2 weeks of treatment with AMD3465 or PBS is shown.

FIGS. 13A-F demonstrate that CXCR4 regulates a T cell-specific gene signature and promotes LIC activity in T-ALL. FIG. 13A is a heat map displaying differentially expressed genes in Cxcr4^{f/f} and Cxcr4^{f/f} Mx1-Cre T-ALL cells 10 days after administration of poly(I:C). FIG. 13B is a scatter plot comparing log₂-transformed FPKM expression values for CXCR4-wildtype and -deficient T-ALL cells. Each dot represents an individual gene. Red dots represent genes of interest over-expressed in CXCR4-wildtype (y-axis) compared to CXCR4-deficient T-ALL cells (x-axis). FIG. 13C shows Myc expression signatures are enriched in T-ALL cells with intact CXCR4 signaling as determined by GSEA. The top shows a comparison Cxcr4^{f/f} and Cxcr4^{f/f} Mx1-Cre T-ALL cells. The bottom shows a comparison of T-ALL cells treated with AMD3100 or vehicle for 4 days in vitro. FIG. 13D shows MYC-GFP expression by Fbxw7 mut Notch1-ΔE LICs expressing a Myc-GFP fusion allele, 4 days after culture on OP9 cells in media with AMD3465 or vehicle, measured by flow cytometry. The graph represents 3 experiments. FIGS. 13E-F show Cxcr4^{f/f} Mx1-Cre or control Cxcr4^{+/+} Mx1-Cre Notch1-ΔE⁺ T-ALL cells treated with poly(I:C) in vivo isolated from spleen and bone marrow and transferred into sublethally irradiated wild-type mice (1 million cells per mouse; n=3 recipients per genotype). FIG. 13E is an image showing spleens and lymph nodes harvested from secondary recipients 10 weeks after transplantation. FIG. 13F shows the cell number of Cxcr4^{f/f} Mx1-Cre or control Cxcr4^{+/+}Mx1-Cre T-ALL cells in indicated tissues of secondary recipients 10 weeks after transplantation, assessed by flow cytometry. Bars represent the mean.

FIGS. 14A-G demonstrate that CXCR4 promotes MYC expression in T-ALL cells. FIG. 14A is a heat map depicting differentially expressed genes (Myc targets are indicated) in primary mouse T-ALL cells after 4 day culture with OP9 cells in the presence of AMD3100 or vehicle (right; n=3). FIG. 14B shows Myc expression signatures are enriched in T-ALL cells with intact CXCR4 signaling (vehicle-treated), as determined by GSEA using previously published gene sets (Kim et al., “An Extended Transcriptional Network for Pluripotency of Embryonic Stem Cells,” Cell 132:1049-1061 (2008); King et al., “The Ubiquitin Ligase FBXW7 Modulates Leukemia-initiating Cell Activity by Regulating MYC Stability,” Cell 153:1552-1566 (2013); Schuhmacher et al., “The Transcriptional Program of a Human B Cell Line in Response to Myc,” Nucl. Acids Res. 29:397-406 (2001), which are hereby incorporated by reference in their entirety). FIG. 14C shows Myc protein expression in Cxcr4^{f/f} Mx1-Cre or control Cxcr4^{+/+} Mx1-Cre T-ALL cells purified from liver and spleen after poly(I:C) treatment. Data represents 3 different experiments, with cells pooled from 1-3 mice per experiment. FIG. 14D shows Myc expression in purified bone marrow T-ALL cells 8 hours after subcutaneous administration of AMD3465 (2 doses, 25 mg/kg, 4 hours apart) or vehicle (PBS). Data represents 3 mice per group. FIGS. 14E-F show Myc expression and Myc target expression in purified spleen T-ALL cells from control and VEcad-cre;Cxcl12^{fl/fl} 4 weeks post-transplantation. Western blot (FIG. 14E) represents cells pooled from 1-4 mice, in one experiment. RT-qPCR (FIG. 14F) compiles data from at least 3 mice per group. Error bars represent +/−SD. FIG. 14G shows mouse T-ALL cells from spleen transduced with MIG-IRES-GFP-cMyc or MIG-IRES-GFP (control), purified on the basis of GFP expression, then co-cultured with OP9 cells for 4 days in the presence of the indicated doses of AMD3465. Data represents 1 experiment with 3 replicates per group for each AMD3465 concentration tested. Error bars represent +/−SD.

### DETAILED DESCRIPTION OF THE INVENTION

A first aspect of the present invention is directed to a method of treating T-cell acute lymphoblastic leukemia in a subject. This method involves selecting a subject having T-cell acute lymphoblastic leukemia and administering, to the selected subject, a therapeutic agent that inhibits CXCR4-CXCL12 signaling at a dosage effective to treat the T-cell acute lymphoblastic leukemia in the subject.

As used herein, “subject” refers to any animal having T-cell acute lymphoblastic leukemia that is amenable to treatment in accordance with the methods of the present invention. Preferably, the subject is a mammal. Exemplary mammalian subjects include, without limitation, humans, non-human primates, dogs, cats, rodents (e.g., mouse, rat, guinea pig), horses, cattle and cows, sheep, and pigs.

As used herein, “T-cell acute lymphoblastic leukemia” (T-ALL) refers to an aggressive tumor of immature T cells that is characterized by infiltration of the bone marrow and blood with lymphoblasts of the T lymphoid phenotype and to all subtypes of the disease including, but not limited to, adult T-cell acute lymphoblastic leukemia, pediatric T-cell acute lymphoblastic leukemia, and early T-cell precursor acute lymphoblastic leukemia. T-ALL is distinct from adult T-cell leukemia/lymphoma, which is a malignancy of mature T cells cause by human T-cell lymphotrophic virus type I.

Therapeutic agents of the present invention are agents that inhibit the chemokine receptor-chemokine interaction of CXCR4-CXCL12 and/or the subsequent molecular signaling pathway triggered by the CXCR4-CXCL12 interaction.

Chemokines, including CXCL12, function at least in part, by modulating a complex and overlapping set of biological activities important for the movement of lymphoid cells, and extravasation and tissue infiltration of leukocytes in response to inciting agents. These chemotactic cytokines, or chemokines, constitute a family of proteins, approximately 8-10 kDa in size, that are expressed by a wide variety of cells, to attract macrophages, T-cells, eosinophils, basophils, and neutrophils to sites of inflammation, and play a role in the maturation of cells of the immune system. CXCL12 is known to play a role in many diverse cellular functions, including embryogenesis, immune surveillance, inflammation response, tissue homeostasis, and tumor growth and metastasis. CXCL12 is the ligand to chemokine receptor CXCR4. The nucleotide and amino acid sequences of the human CXCL12 are well known in the art, see for example, NCBI Reference Sequences CR450283.1 and AAV49999.1, respectively, and which are hereby incorporated by reference in their entirety.

Chemokine receptors are cell-surface receptors belonging to the family of G-protein-coupled seven-transmembrane proteins that mediate the biological activity of chemokines. Chemokine receptors are classified based upon the chemokine that constitutes the receptor's natural ligand. A multitude of chemokine receptors have been characterized and are well known in the art. CXCR4, the chemokine receptor for CXCL12, is broadly expressed on cells of both the immune and central nervous systems. The nucleotide and amino acid sequences of the human CXCR4 are well known in the art, see for example, NCBI Reference Sequences AF025375.1 and P61073.1, respectively, which are hereby incorporated by reference in their entirety.

A therapeutic agent of the present invention includes any agent that inhibits CXCR4-CXCL12 signaling. Suitable agents include agents that inhibit CXCR4-CXCL12 signaling directly by inhibiting CXCR4-CXCL12 binding interaction. Alternatively, suitable therapeutic agents include agents that inhibit CXCR4-CXCL12 signaling indirectly by inhibiting the downstream molecular signaling pathway that is triggered subsequent the CXCR4-CXCL12 interaction.

In one embodiment of the present invention, the therapeutic agent is an inhibitor of CXCR4. In another embodiment of the present invention, the therapeutic agent is an inhibitor of CXCL12. Suitable CXCL12 inhibitors and CXCR4 inhibitors include nucleic acid inhibitor molecules, inhibitory peptides, antibodies, or small molecules as described herein.

In one embodiment, the CXCR4 inhibitor is a small molecule. Small molecule inhibitors of CXCR4 have been described in the art and are often cationic molecules able to bind the predominantly anionic extracellular domain of CXCR4 (Debnath et al., “Small Molecule Inhibitors of CXCR4,” Theranostics 3(1):47-75 (2013), which is hereby incorporated by reference in its entirety). Numerous small molecule CXCR4 antagonists have been identified including, without limitation, AMD 3100 (Plerixafor; 1,1′-[1,4-phenylenebis(methylene)]bis[1,4,8,11-tetraazacyclotetradecane]) (De Clercq et al., “Highly Potent and Selective Inhibition of Human Immunodeficiency Virus by the Bicyclam Derivative JM3100,” Antimicrob. Agents Chemother. 38:668-674 (1994); U.S. Pat. No. 5,583,131 to Bridger et al, which are hereby incorporated by reference in their entirety), which is a bicyclam derivative, and its derivatives such as AM070 (Skerlj et al., “Discovery of Novel Small Molecule Orally Bioavailable C—X—C Chemokine Receptor 4 Antagonists That are Potent Inhibitors of T-tropic (×4) HIV-1 Replication,” J. Med. Chem. 53:3376-3388 (2010), which is hereby incorporated by reference in its entirety). Other known small molecule inhibitors of CXCR4 include N[1,4,8,11-tetraazacyclotetradecanyl-1,4-phenylene-bis-(methylene)]-2-aminomethyl-pyridine (AMD3465), which is an N-pyridinylmethylene cyclam (Hatse et al., “AMD3465, a monomacrocyclic CXCR4 antagonist and potent HIV entry inhibitor,” Biochem. Pharmacol. 70:752-761 (2005), which is hereby incorporated by reference in its entirety), N,N′-(1,4-phenylenebis(methylene))dipyrimidine-2-amine (MSX-122) (Liang et al., “Development of a Unique Small Molecule Modulator of CXCR4,” PloS ONE 7(4):e34038 (2012), which is hereby incorporated by reference in its entirety), and TG-0054 (Huang et al., “Rapid Mobilization of Murine Hematopoietic Stem and Progenitor Cells with TG-0054, a Novel CXCR4 Antagonist,” Blood (ASH Annual Meeting Abstracts) 114:866 (2009), which is hereby incorporated by reference in its entirety). Suitable small molecule CXCR4 inhibitors are further disclosed in WO 2010/025416, which is hereby incorporated by reference in its entirety.

In another embodiment, the CXCR4 inhibitor is an inhibitory peptide. Peptide inhibitors of CXCR4 have been described in the art and are often cationic molecules able to bind the predominantly anionic extracellular domain of CXCR4 (Debnath et al., “Small Molecule Inhibitors of CXCR4,” Theranostics 3(1):47-75 (2013), which is hereby incorporated by reference in its entirety). One exemplary inhibitor peptide is known as T140, having an amino acid sequence of H-Arg-Arg-Nal-Cys-Tyr-Arg-Lys-dLys-Pro-Tyr-Arg-Cit-Cys-Arg-OH (SEQ ID NO: 4), where Cit is L-citrulline, Nal is L-3-(2-napthyl)alanine, and a disulfide links the two Cys residues (Tamamura et al., “Pharmacophore Identification of a Specific CXCR4 Inhibitor, T140, Leads to Development of Effective Anti-HIV Agents With Very High Selectivity Indexes,” Bioorg. Med. Chem. Lett. 10:2633-2637 (2000), which is hereby incorporated by reference in its entirety). Derivatives of T140 have also been described including, without limitation, TN14003, which comprises the amino acid sequence of H-Arg-Arg-Nal-Cys-Tyr-Cit-Lys-DLys-Pro-Tyr-Arg-Cit-Cys-Arg-NH2 (SEQ ID NO: 5), where Cit is L-citrulline, Nal is L-3-(2-napthyl)alanine, and a disulfide links the two Cys residues (Tamamura et al., “Development of Specific CXCR4 Inhibitors Possessing High Selectivity Indexes as Well as Complete Stability in Serum Based on an Anti-HIV Peptide T140,” Bioorg. Med. Chem. Lett. 11:1897-1902 (2001), which is hereby incorporated by reference in its entirety), BKT140 (4F-benzoyl-TN14003) (Peled et al., “The High Affinity CXCR4 Antagonist BKT140 is Safe and Induces a Robust Mobilization of Human CD34+ Cells in Patients With Multiple Myeloma,” Clin. Cancer. Res. 20(2):469-479 (2014), which is hereby incorporated by reference in its entirety), FC131 (Thiele et al., Determination of the Binding Mode for the Cyclopentapeptide CXCR4 Antagonist FC131 Using a Dual Approach of Ligand Modifications and Receptor Mutagenesis,” Br. J. Pharmacol. 171(23):5313-5329 (2014), which is hereby incorporated by reference in its entirety), as well as derivatives described in U.S. Patent Application Publication No. US 2016/0082071 to Peled et al., which is hereby incorporated by reference in its entirety. Other exemplary peptide antagonists include, but are not limited to, POL5551 (Karpova et al., “The Novel CXCR4 Antagonist POL5551 Mobilizes Hematopoietic Stem and Progenitor Cells With Greater Efficiency Than Plerixafor,” Leukemia 12:2322-2331 (2013), which is hereby incorporated by reference in its entirety), LY2510924 (Galsky et al., “A Phase I Trial of LY2510924, a CXCR4 Peptide Antagonist, in Patients With Advanced Cancer,” Clin. Cancer Res. 20(13):3581-3588 (2014), which is hereby incorporated by reference in its entirety), Peptide R (Ierano et al., “CXCR4-Antagonist Peptide R-liposomes for Combined Therapy Against Lung Metastasis,” Nanoscale 8(14):7562-7571 (2016), which is hereby incorporated by reference in its entirety), KRH-1636 (Zachariassen et al., “Probing the Molecular Interactions Between CXC Chemokine Receptor 4 (CXCR4) and an Arginine-based Tripeptidomimetic Antagonist (KRH-1636),” J. Med. Chem. 58(20):8141-8153 (2015), which is hereby incorporated by reference in its entirety), and POL6326 (Polyphor) (Rettig et al., “Mobilization of Hematopoietic Stem and Progenitor Cells Using Inhibitors of CXCR4 and VLA-4,” Leukemia 26(1):34-53 (2012), which is hereby incorporated by reference in its entirety).

In another embodiment, the inhibitor is a CXCL12 small molecule inhibitor. By way of example, compounds belonging to the family of chalcones have been identified as inhibiting binding of CXCL12 to CXCR4. These compounds include, (E)-1,3-diphenylprop-2-en-1-one, (E)-3-(4-hydroxyphenyl)-1-phenylprop-2-en-1-one, (E)-1-(6′-hydroxyphenyl)-3-(4-hydroxyphenyl)prop-2-en-1-one, and (E)-1-(4′-chlorphenyl)-3-(4-hydroxyphenyl)prop-2-en-1-one (Hachet-Haas et al., “Small Neutralizing Molecules to Inhibit Actions of the Chemokine CXCL12,” J Biol. Chem. 283(34):23189-23199 (2008), which is hereby incorporated by reference in its entirety).

In another embodiment, the inhibitor is a nucleic acid inhibitor of CXCL12. A suitable nucleic acid inhibitor for use in the present invention is NOX-A12, a structured mirror-image RNA oligonucleotide that neutralizes CXCL12 (Hoellenriegel et al., “The Spiegelmer NOX-A12, a Novel CXCL12 Inhibitor, Interferes with Chronic Lymphocytic Leukemia Cell Motility and Causes Chemosensitization,” Blood 123(7) (2014), which is hereby incorporated by reference in its entirety).

In another embodiment, the therapeutic agent comprises an antibody or binding portion thereof that binds to CXCR4 or CXCL12 and inhibits CXCR4-CXCL12 interaction and or signaling activity.

As used herein, the term “antibody” encompasses intact immunoglobulins derived from natural sources or from recombinant sources, as well as immunoreactive portions (i.e. antigen binding portions) of intact immunoglobulins. The antibodies of the present invention may exist in a variety of forms including, for example, polyclonal antibodies, monoclonal antibodies, intracellular antibodies, antibody fragments (e.g. Fv, Fab and F(ab)2), single chain antibodies (scFv), single-domain antibodies, chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc Natl Acad Sci USA 85:5879-5883 (1988); Bird et al, “Single-Chain Antigen-Binding Proteins,” Science 242:423-426 (1988), which are hereby incorporated by reference in their entirety).

Single-domain antibodies (sdAb; nanobody) are antibody fragments consisting of a single monomeric variable antibody domain (˜12-15 kDa). The sdAb are derived from the variable domain of a heavy chain (VH) or the variable domain of a light chain (VL). sdAbs can be naturally produced, i.e., by immunization of dromedaries, camels, llamas, alpacas or sharks (Ghahroudi et al., “Selection and Identification of Single Domain Antibody Fragments from Camel Heavy-Chain Antibodies,” FEBS Letters 414(3): 521-526 (1997), which is hereby incorporated by reference in its entirety). Alternatively, the antibody can be produced in microorganisms or derived from conventional whole antibodies (Harmsen et al., “Properties, Production, and Applications of Camelid Single-Domain Antibody Fragments,” Appl. Microbiol. Biotechnology 77:13-22 (2007), Holt et al., “Domain Antibodies: Proteins for Therapy,” Trends Biotech. 21(11): 484-490 (2003), which is hereby incorporated by reference in its entirety).

Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (i.e., CXCR4 receptor or CXCL12 ligand) either in vivo or in vitro.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur J Immunol 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

In another embodiment of the present invention, monoclonal antibodies or antibody fragments can be isolated from antibody phage libraries generated using the techniques described in McCafferty et al., “Phage Antibodies: Filamentous Phage Displaying Antibody Variable Domains,” Nature 348:552-554 (1990), which is hereby incorporated by reference in its entirety. Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety, describe the isolation of murine and human antibodies, respectively, using phage libraries. Subsequent publications describe the production of high affinity (nM range) human antibodies by chain shuffling (Marks et al., BioTechnology 10:779-783 (1992), which is hereby incorporated by reference in its entirety), as well as combinatorial infection and in vivo recombination as a strategy for constructing very large phage libraries (Waterhouse et al., Nuc. Acids. Res. 21:2265-2266 (1993), which is hereby incorporated by reference in its entirety). Thus, these techniques are viable alternatives to traditional monoclonal antibody hybridoma techniques for isolation of monoclonal antibodies.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, generate monoclonal antibodies.

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

“Humanized” forms of non-human (e.g., rodent) antibodies are chimeric antibodies that contain minimal sequences derived from the non-human antibody. For the most part, humanized antibodies are human immunoglobulins (recipient antibody) in which residues from a hypervariable region are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992), which are hereby incorporated by reference in their entirety.

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers by substituting hypervariable region sequences for the corresponding sequences of a human antibody (Jones et al., Nature 321:522-525 (1986); Reichmann et al., Nature 332:323-327 (1988); Verhoeyen et al., Science 239:1534-1536 (1988), which are hereby incorporated by reference in their entirety). Accordingly, such “humanized” antibodies are chimeric antibodies wherein substantially less than an intact human variable domain has been substituted by the corresponding sequence from a non-human species (see U.S. Pat. No. 4,816,567, which is hereby incorporated by reference in its entirety). In practice, humanized antibodies are typically human antibodies in which some hypervariable region residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol. 196:901 (1987), which are hereby incorporated by reference in their entirety). Another method uses a particular framework region derived from the consensus sequence of all human antibodies of a particular subgroup of light or heavy chains. The same framework may be used for several different humanized antibodies (Carter et al., Proc. Natl. Acad Sci. USA 89:4285 (1992); Presta et al, J. Immunol. 151:2623 (1993), which are hereby incorporated by reference in their entirety).

It is further important that antibodies be humanized with retention of high binding affinity for the antigen and other favorable biological properties. To achieve this goal, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, and an analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of humanized anti-CXCR4 and anti-CXCL12 antibodies are contemplated. For example, the humanized antibody may be an antibody fragment, such as a Fab. Alternatively, the humanized antibody may be an intact antibody, such as an intact IgG1 antibody. An exemplary humanized anti-CXCR4 antibody suitable for the treatment of T-ALL as described herein is disclosed in Kuhne et al., “BMS-936564/MDX-1338: A Fully Human Anti-CXCR4 Antibody Induces Apoptosis In Vitro and Shows Antitumor Activity In Vivo in Hematologic Malignancies,” Clin. Cancer Res. 19(2): 357-366 (2013), which is hereby incorporated by reference in its entirety. An exemplary humanized anti-CXCL12 antibody suitable for the treatment of T-ALL as described herein is disclosed in Zhong et al., “Development and Preclinical Characterization of a Humanized Antibody Targeting CXCL12,” Clin. Cancer Res. 19(16):4433-45 (2013), which is hereby incorporated by reference in its entirety.

As an alternative to humanization, human antibodies can be generated. For example, transgenic animals (e.g., mice) that are capable, upon immunization, of producing a full repertoire of human antibodies in the absence of endogenous immunoglobulin production can be utilized for human antibody production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl Acad. Sci. USA 90:2551 (1993); Jakobovits et al., Nature 362:255-258 (1993); U.S. Pat. No. 5,545,806 to Lonberg et al, U.S. Pat. No. 5,569,825 to Lonberg et al, and U.S. Pat. No. 5,545,807 to Surani et al, which are hereby incorporated by reference in their entirety.

Alternatively, phage display technology (McCafferty et al., Nature 348:552-553 (1990), which is hereby incorporated by reference in its entirety) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, see e.g., Johnson and Chiswell, Current Opinion in Structural Biology 3:564-571 (1993), which is hereby incorporated by reference in its entirety. Several sources of V-gene segments can be used for phage display (see e.g., Clackson et al., Nature 352:624-628 (1991), which is hereby incorporated by reference in its entirety). A repertoire of V genes from unimmunized human donors can be constructed and antibodies to a diverse array of antigens (including self-antigens) can be isolated essentially following the techniques described by Marks et al., J. Mol. Biol. 222:581-597 (1991), Griffith et al., EMBO J. 12:725-734 (1993), see e.g., U.S. Pat. No. 5,565,332 to Hoogenboom and U.S. Pat. No. 5,573,905 to Lerner et al., which are hereby incorporated by reference in their entirety.

In addition to whole antibodies, the present invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

Antibody fragments can also be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992), which is hereby incorporated by reference in its entirety). According to another approach, F(ab′)₂ fragments can be isolated directly from recombinant host cell culture. Fab and F(ab′)₂ fragments with increased in vivo half-life comprising a salvage receptor binding epitope residues are described in U.S. Pat. No. 5,869,046 to Presta, which is hereby incorporated by reference in its entirety. Other techniques for the production of antibody fragments will be apparent to the skilled practitioner. In other embodiments, the antibody of choice is a single chain Fv fragment (scFv) (see U.S. Pat. No. 5,571,894 to Wels and U.S. Pat. No. 5,587,458 to King et al, which are hereby incorporated by reference in their entirety). Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv.

Bispecific antibodies are antibodies that have binding specificities for at least two different epitopes. Exemplary bispecific antibodies may bind to two different epitopes of the CXCR4 receptor protein or CXCL12 protein. Alternatively, such antibodies may combine a CXCR4 or CXCL12 binding sites with a binding site for another protein, for example, a T-ALL cell specific surface protein to target CXCR4 antibody binding to T-ALL cells or an endothelial cell specific surface protein to target CXCL12 antibody binding to endothelial cells. Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety). Generally, bispecific antibodies are secreted by triomas (i.e., lymphoma cells fuse to a hybridoma) and hybrid hybridomas. The supernatants of triomas and hybrid hybridomas can be assayed for bispecific antibody production using a suitable assay (e.g., ELISA), and bispecific antibodies can be purified using conventional methods. These antibodies can then be humanized according to methods known in the art. Humanized bispecific antibodies or a bivalent antigen-binding fragment of the bispecific antibody having binding specificity for CXCR4 or CXCL12 proteins and an antigen expressed on a target T-ALL or other cancer cell, provides a cell-specific targeting approach.

In one embodiment of the present invention, the therapeutic agent is a CXCR4 antibody or binding portion thereof. A CXCR4 antibody of the present invention encompasses any immunoglobulin molecule as described above that specifically binds to an epitope of CXCR4 and inhibits CXCR4 mediated signaling. As used herein, “epitope” refers to a region of CXCR4 that is recognized by the isolated antibody and involved in mediating interaction with CXCL12 or the downstream molecular signaling pathway triggered by CXCL12-CXCR4 binding interaction.

Suitable antagonist or inhibitory antibodies of CXCR4 recognize and bind to epitopes within one or more of the three extracellular loops (ECL) of CXCR4 and/or the N-terminal region of CXCR4. Suitable epitopes include those within ECL1, which corresponds to amino acid residues 97-110 of human CXCR4 having the amino acid sequence of SEQ ID NO: 1 (NCBI Reference Sequence P61073.1) (shown below). Alternatively, suitable epitopes include those within ECL2, which corresponds to amino acids 176-202 of SEQ ID NO: 1, and those within ECL3, which corresponds to amino acids 262-282 of SEQ ID NO: 1. Other epitopes include those in the N-terminal region of CXCR4, which corresponds to amino acids 1-39 of SEQ ID NO: 1.

```
Human CXCR4
SEQ ID NO: 1
Met Glu Gly Ile Ser Ile Tyr Thr Ser Asp Asn Tyr
1 5 10
Thr Glu Glu Met Gly Ser Gly Asp Tyr Asp Ser Met
                15 20
Lys Glu Pro Cys Phe Arg Glu Glu Asn Ala Asn Phe
25 30 35
Asn Lys Ile Phe Leu Pro Thr Ile Tyr Ser Ile Ile
                        40 45
Phe Leu Thr Gly Ile Val Gly Asn Gly Leu Val Ile
        50 55 60
Leu Val Met Gly Tyr Gln Lys Lys Leu Arg Ser Met
                                65 70
Thr Asp Lys Tyr Arg Leu His Leu Ser Val Ala Asp
                75 80
Leu Leu Phe Val Ile Thr Leu Pro Phe Trp Ala Val
85 90 95
Asp Ala Val Ala Asn Trp Tyr Phe Gly Asn Phe Leu
                        100 105
Cys Lys Ala Val His Val Ile Tyr Thr Val Asn Leu
        110 115 120
Tyr Ser Ser Val Leu Ile Leu Ala Phe Ile Ser Leu
                                125 130
Asp Arg Tyr Leu Ala Ile Val His Ala Thr Asn Ser
                135 140
Gln Arg Pro Arg Lys Leu Leu Ala Glu Lys Val Val
145 150 155
Tyr Val Gly Val Trp Ile Pro Ala Leu Leu Leu Thr
                        160 165
Ile Pro Asp Phe Ile Phe Ala Asn Val Ser Glu Ala
        170 175 180
Asp Asp Arg Tyr Ile Cys Asp Arg Phe Tyr Pro Asn
                                185 190
Asp Leu Trp Val Val Val Phe Gln Phe Gln His Ile
                195 200
Met Val Gly Leu Ile Leu Pro Gly Ile Val Ile Leu
205 210 215
Ser Cys Tyr Cys Ile Ile Ile Ser Lys Leu Ser His
                        220 225
Ser Lys Gly His Gln Lys Arg Lys Ala Leu Lys Thr
        230 235 240
Thr Val Ile Leu Ile Leu Ala Phe Phe Ala Cys Trp
                                245 250
Leu Pro Tyr Tyr Ile Gly Ile Ser Ile Asp Ser Phe
                255 260
Ile Leu Leu Glu Ile Ile Lys Gln Gly Cys Glu Phe
265 270 275
Glu Asn Thr Val His Lys Trp Ile Ser Ile Thr Glu
                        280 285
Ala Leu Ala Phe Phe His Cys Cys Leu Asn Pro Ile
        290 295 300
Leu Tyr Ala Phe Leu Gly Ala Lys Phe Lys Thr Ser
                                305 310
Ala Gln His Ala Leu Thr Ser Val Ser Arg Gly Ser
                315 320
Ser Leu Lys Ile Leu Ser Lys Gly Lys Arg Gly Gly
325 330 335
His Ser Ser Val Ser Thr Glu Ser Glu Ser Ser Ser
                        340 345
Phe His Ser Ser
        350
```

Suitable CXCR4 antagonist antibodies for use in the methods as described herein are know in the art and include, without limitation, BMS-936564/MDX1338 (Kuhne et al., “BMS-936564/MDX-1338: a Fully Human Anti-CXCR4 Antibody Induces Apoptosis In Vitro and Shows Antitumor Activity In Vivo in Hematologic Malignancies,” Clin. Cancer. Res. 19(2):357-66 (2012), which is hereby incorporated by reference in its entirety), MEDI3185 (Peng et al., “Molecular Basis for the Antagonistic Activity of an Anti-CXCR4 Antibody,” mAbs 8:163-175 (2016), which is hereby incorporated by reference in its entirety), LY2624587 (Peng et al., “Inhibition of CXCR4 by LY2624587, a Fully Humanized Anti-CXCR4 Antibody Induces Apoptosis of Hematologic Malignancies,” PLoS ONE 11(3):e0150585 (2016), which is hereby incorporated by reference in its entirety), 1A4 (Li et al., “Preparation and Characterization of a New Monoclonal Antibody Against CXCR4 Using Lentivirus Vector,” Int. Immunopharmacol. 36:100-105 (2016), which is hereby incorporated by reference in its entirety), 12G5 (Carnec et al., “Anti-CXCR4 Monoclonal Antibodies Recognizing Overlapping Epitopes Differ Significantly in Their Ability to Inhibit Entry of Human Immunodeficiency Virus Type 1,” J. Virol. 79(3):1930-1933 (2005), which is hereby incorporated by reference in its entirety), A145 and A120 (Tanaka et al., “Unique Monoclonal Antibody Recognizing the Third Extracellular Loop of CXCR4 Induces Lymphocyte Agglutination and Enhances Human Immunodeficiency Virus Type 1-Mediated Syncytium Formation and productive Infection,” J. Virol. 75(23):11534-11543 (2001), which is hereby incorporated by reference in its entirety), and 238D2 and 238D4 nanobodies (Jahnichen et al., “CXCR4 Nanobodies (VHH-based Single Variable Domains) Potently Inhibit Chemotaxis and HIV-1 Replication and Mobilize Stem Cells,” PNAS 107(47):20565-20570 (2010), which is hereby incorporated by reference in its entirety).

In another embodiment of the present invention, the therapeutic agent is a CXCL12 antibody or binding portion thereof. A CXCL12 antibody or binding portion thereof the present invention encompasses any immunoglobulin molecule that specifically binds to an epitope of CXCL12 involved in mediating interaction with CXCR4 or the downstream molecular signaling pathway triggered by CXCL12-CXCR4 binding interaction.

In one embodiment, suitable CXCL2 antibodies for use in the methods described herein include those that bind an epitope that corresponds to amino acid residues Asn65 and Asn66 of human CXCL12 having the amino acid sequence of SEQ ID NO: 2 (NCBI Reference Sequence AAV49999.1) as shown below. In another embodiment, a suitable CXCL2 antibody binds an epitope within or comprising the RFFESH (SEQ ID NO: 3) fragment near the N-terminus of SEQ ID NO: 2, which is involved in binding to CXCR4 (Crump et al., “Solution Structure and Basis for Functional Activity of Stromal Cell-derived Factor-1; Dissociation of CXCR4 Activation From Binding and Inhibition of HIV-1,” EMBO J. 16:6996-7007 (1997), which is hereby incorporated by reference in its entirety).

```
Human CXCL12
SEQ ID NO: 2
Met Asn Ala Lys Val Val Val Val Leu Val Leu Val
1 5 10
Leu Thr Ala Leu Cys Leu Ser Asp Gly Lys Pro Val
                15 20
Ser Leu Ser Tyr Arg Cys Pro Cys Arg Phe Phe Glu
25 30 35
Ser His Val Ala Arg Ala Asn Val Lys His Leu Lys
                        40 45
Ile Leu Asn Thr Pro Asn Cys Ala Leu Gln Ile Val
        50 55 60
Ala Arg Leu Lys Asn Asn Asn Arg Gln Val Cys Ile
                                65 70
Asp Pro Lys Leu Lys Trp Ile Gln Glu Tyr Leu Glu
                75 80
Lys Ala Leu Asn Lys Arg Phe Lys Met
85 90
```

A CXCL12 antagonist antibody that is suitable for use in the methods described herein is described by Zhong et al., “Development and Preclinical Characterization of a Humanized Antibody Targeting CXCL12,” Clin. Cancer. Res. 19(16):4433-4445 (2013), which is hereby incorporated by reference in its entirety.

Antibody modifications that enhance stability or facilitate delivery of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol.

In another embodiment, the CXCR4 and/or CXCL12 inhibitor is coupled to a targeting moiety to achieve targeted delivery of the CXCR4 and/or CXCL12 inhibitor to the leukemic or vascular cells of interest. For example, as described in the Examples herein, CXCL12 in vascular endothelial cells, but not perivascular cells, is necessary for T-ALL progression. Accordingly, the CXCL12 inhibitors can be coupled to an endothelial or vascular targeting moiety, such as, for example and without limitation, an anti-ICAM-1 antibody or anti-PECAM-1 antibody. These carriers are particularly attractive because endothelial cells do not internalize the PECAM-1 and ICAM-1 antibodies; however, endothelial cell internalize very effectively multimolecular and multivalent complexes of proteins, nucleic acids, and polymeric nanocarriers conjugated to these antibodies via the CAM-mediated endocytic pathway (see Muzykantov and Muro, “Targeting Delivery of Drugs in the Vascular System,” Int. J. Transp. Phenom. 12(1-2): 41-49 (2011), which is hereby incorporated by reference in its entirety). In accordance with this embodiment, and in embodiments not involving targeted delivery of the CXCR4 and CXCL12 inhibitory agents described herein, the inhibitory agents may be encapsulated in or conjugated to an appropriate carrier molecule, such as, e.g., a liposome, polymersome, magnetic nanoparticle, perfluorocarbon nanoparticle, dendrimer, and/or quantom dot (see Muzykantov V R, “Targeted Drug Delivery to Endothelial Adhesion Molecules,” ISRN Vacular Medicine, vol. 2013, 916254 (2013), which is hereby incorporated by reference in its entirety).

In one embodiment, the method involves selecting a subject having T-cell acute lymphoblastic leukemia and administering a chemotherapeutic agent in combination with the therapeutic agent that inhibits CXCR4-CXCL12 signaling at a dosage effective to treat T-cell acute lymphoblastic leukemia in the selected subject.

In accordance with this aspect of the present invention, the chemotherapeutic agent is selected from the group consisting of cytarabine, vincristine, prednisone, doxorubicin, daunorubicin, PEG asparaginase, methotrexate, cyclophosphamide, L-asparaginase, etoposide, and leucovorin.

In a further embodiment, the therapeutic agent that inhibits CXCR4-CXCL12 is administered in combination with a Notch-1 antagonist. Because activation of the Notch-1 signaling pathway has been observed in over 80% of all T-cell acute lymphoblastic leukemia cases, a number of Notch-1 antagonists have been developed and are well known in the art. Suitable Notch-1 antagonists include without limitation gamma-secretase inhibitors selected from the group consisting of [(2S)-2-{[(3,5-Difluorophenyeacetyl]amino}-N-[(3 S)1-methyl-2-oxo-5-phenyl-2,3-dihydro-1H-1,4-benzodiazepin-3-yl]propanamide] (CompE), N4N-(3,5-difluorophenacetyl)-L-alanyl]-Sphenylglycine-t-butylester (DAPT), LY411575, (5 S)-(t-Butoxycarbonylamino)-6-phenyl-(4R)hydroxy-(2R)benzylhexanoyl)-L-leu-L-phe-amide (L-685,458), L-852,647, MW167, WPE-111-31, LY450139, MRK003, R-flurbiprofen ([1,1′-Biphenyl]-4-acetic acid, 2-fluoro-alpha-methyl), NGX-555, CZC-1040, E2012, GSI-1, Begacestat (2-Thiophenesulfonamide, 5-chloro-N-[(1 S)-3₁3,3-trifluoro-1-(hydroxymethyl)-2-(trifluoromethyl)propyl]-), NIC5-15, BACE Inhibitor, and CHF-5074.

The therapeutic agents of the present invention can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Typically, parenteral administration is the preferred mode of administration.

Therapeutic agents of the present invention are formulated in accordance with their mode of administration. For oral administration, for example, the therapeutic agents of the present invention are formulated into an inert diluent or an assimilable edible carrier, enclosed in hard or soft shell capsules, compressed into tablets, or incorporated directly into food. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits inhibition of proteolysis and uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, “Soluble Polymer-Enzyme Adducts,” In: Enzymes as Drugs, Hocenberg and Roberts, eds., Wiley-Interscience (1981), which is hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

The therapeutic agents of the present invention may also be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutic agents of the present invention vary depending upon many different factors, including type and stage of leukemia, mode of administration, target site, physiological state of the patient, other medications or therapies administered, and physical state of the patient relative to other medical complications. Treatment dosages need to be titrated to optimize safety and efficacy.

In a preferred embodiment, the administering step is repeated periodically as needed (e.g., hourly, daily, weekly, monthly, yearly).

The CXCR4 and CXCL12 therapeutic agents of the present invention can be administered in a single dose or multiple doses. The dosage can be determined by methods known in the art and can be dependent, for example, upon the individual's age, sensitivity, tolerance and overall well-being. Suitable dosages for antibodies can be from about 0.1 mg/kg body weight to about 10.0 mg/kg body weight per treatment.

The therapeutic agents of the present invention can be administered to an individual (e.g., a human) alone or in conjunction with one or more other therapeutic agents of the invention.

The therapeutic agents of the present invention are also suitable to treat the various stages of T-cell acute lymphoblastic leukemia including, but not limited to, pro-T-ALL, Pre-TALL, cortical T-ALL, and medulla T-ALL, relapsed T-cell acute lymphoblastic leukemia, and refractory T-cell acute lymphoblastic leukemia. “Treating” T-ALL as described herein includes, without limitation, suppressing leukemia cell growth and/or proliferation and/or prolonging the survival of the subject as a result of the administration of the therapeutic agents described herein.

Another aspect of the present invention is directed to a method of inhibiting T-cell acute lymphoblastic leukemia cell proliferation and/or survival. This method involves administering to a population of T-cell acute lymphoblastic leukemia cells an inhibitor of CXCR4-CXCL12 signaling at a dosage effective to inhibit the T-cell acute lymphoblastic leukemia cells proliferation and/or survival.

Suitable therapeutic inhibitors of CXCR4-CXCL12 as well as modes of administration are described supra.

In one embodiment, the inhibitor of CXCR4-CXCL12 signaling is administered to leukemia initiating cells (LICs). As described herein, leukemia initiating cells represent a subset of leukemic cells that possess properties similar to normal hematopoietic stem cells such as self-renewal, quiescence, and resistance to traditional chemotherapy (Bonnet & Dick, “Human Acute Myeloid Leukemia is Organized as a Hierarchy That Originates From a Primitive Hematopoietic Cell,” Nat. Med. 3:730-737 (1997); Huntly & Gilliland, “Leukaemia Stem Cells and the Evolution of Cancer-Stem-Cell Research,” Nat. Rev. Cancer 5:311-321 (2005), which are hereby incorporated by reference in their entirety). As a result, the LIC subset acts as a reservoir of cells contributing to disease, in particular disease relapse. LIC populations have been identified in acute myeloid leukemia, chronic phase and blast crisis CML (Jamieson et al., “Granulocyte-Macrophage Progenitors as Candidate Leukemic Stem Cells In Blast-Crisis CML,” N. Engl. J. Med. 351:657-667 (2004); Sirard et al., “Normal and Leukemic SCID-Repopulating Cells (SRC) Coexist in the Bone Marrow and Peripheral Blood From CML Patients in Chronic Phase, Whereas Leukemic SRC are Detected in Blast Crisis,” Blood 87:1539-1548 (1996); Wang et al., “High Level Engraftment of NOD/SCID Mice by Primitive Normal and Leukemic Hematopoietic Cells From Patients With Chronic Myeloid Leukemia in Chronic Phase,” Blood 91:2406-2414 (1998), which are hereby incorporated by reference in their entirety), and B-cell acute lymphoblastic leukemia (Castro Alves et al., “Leukemia-initiating Cells of Patient-Derived Acute Lymphoblastic Leukemia Xenografts are Sensitive Toward TRAIL,” Blood 119(18):4224-7 (2012), which is hereby incorporated by reference).

In accordance with this aspect of the present invention, the CXCR4-CXCL12 inhibitor can be administered in vivo or in vitro to inhibit T-cell acute lymphoblastic leukemia cell proliferation and/or survival. Administration can be repeated periodically as needed (e.g., hourly, daily, weekly, monthly, yearly) to inhibit T-cell acute lymphoblastic leukemia cell proliferation and/or survival.

### EXAMPLES

The following examples are provided to illustrate embodiments of the present invention but they are by no means intended to limit its scope

### Material and Methods for Examples 1-7

Animals.

Female C57BL/6 mice (6-8 weeks old) were obtained from the National Cancer Institute. Nonobese diabetic/severe combined immunodeficient NOD/MrkBomTac-Prkdc^{scid} (NOD-SCID) were from Taconic. Mx1-Cre (Kuhn et al., “Inducible Gene Targeting in Mice,” Science 269:1427-1429 (1995), which is hereby incorporated by reference in its entirety), VEcad-Cre (B6.FVB-Tg(Cdh5-cre)7Mlia/J) (Alva et al., “VE-Cadherin-Cre-recombinase Transgenic Mouse: a Tool for Lineage Analysis and Gene Deletion in Endothelial Cells,” Dev. Dyn. 235:759-767 (2006), which is hereby incorporated by reference in its entirety), Leprcre (B6.129-Leprtm2(cre)Rck/J) (DeFalco et al., “Virus-assisted Mapping of Neural Inputs to a Feeding Center in the Hypothalamus,” Science 291:2608-2613 (2001), which is hereby incorporated by reference in its entirety), Co12.3-cre (B6.Cg-Tg(Col1a1-cre/ERT2)1Crm/J) (Kim et al., “Transgenic Mice Expressing a Ligand-inducible Cre Recombinase in Osteoblasts and Odontoblasts: a New Tool to Examine Physiology and Disease of Postnatal Bone and Tooth,” Am. J. Pathol. 165:1875-1882 (2004), which is hereby incorporated by reference in its entirety) and Rosa26-Tomato (B6.Cg-Gt(ROSA)26Sortm9(CAG tdTomato)Hze/J) (Madisen et al., “A Robust and High-throughput Cre Reporting and Characterization System for the Whole Mouse Brain,” Nature Neurosci. 13:133-140 (2010), which is hereby incorporated by reference in its entirety) mice were from Jackson laboratories. Cxcr4^{f/f} (Nie et al., “The Role of CXCR4 in Maintaining Peripheral B Cell Compartments and Humoral Immunity,” J. Exp. Med. 200:1145-1156 (2004), which is hereby incorporated by reference in its entirety) Cxcl12^{DsRed} knock-in (Ding and Morrison, “Haematopoietic Stem Cells and Early Lymphoid Progenitors Occupy Distinct Bone Marrow Niches,” Nature 495:231-235 (2013), which is hereby incorporated by reference in its entirety), MycGFP (Huang et al., “Dynamic Regulation of c-Myc Proto-oncogene Expression During Lymphocyte Development Revealed by a GFP-c-Myc Knock-in Mouse,” Eur. J. Immunol. 38:342-349 (2008), which is hereby incorporated by reference in its entirety) and Fbxw7 knock-in mutant mice (King et al., “The Ubiquitin Ligase FBXW7 Modulates Leukemia-initiating Cell Activity by Regulating MYC Stability,” Cell 153:1552-1566 (2013), which is hereby incorporated by reference in its entirety) have previously been described. To induce deletion by Mx1-Cre, mice received three intraperitoneal injections of poly(I:C) (10 ug/g, GE Healthcare) in PBS, administered every other day. Mice were housed in specific pathogen-free conditions at the Skirball Institute animal facility. All animal experiments were performed in accordance with protocols approved by the New York University Institutional Animal Care and Use Committee.

Cell Isolation and Flow Cytometry.

For serial bleeding, blood was collected from the lateral saphenous vein. Following euthanasia, blood was collected via cardiac puncture. Bone marrow cells were harvested by flushing tibias and femurs with PBS supplemented with 2% heat-inactivated fetal bovine serum (FBS; Hyclone). Lymphocytes were isolated from thymus, spleen, lymph nodes, lung, liver and brain by mechanical disruption and filtration through a 100 uM cell strainer (BD Biosciences). Brain and liver lymphocytes were further purified by centrifugation using a 33% isotonic Percoll density gradient (GE Healthcare). Red blood cells were depleted using tris-ammonium chloride solution. Total lymphocytes were counted using a Multisizer 3 (Beckman Coulter) set to detect nuclei between 3.5-10 um. For niche cell isolation, bone marrow was flushed PBS+2% bovine serum. Then whole bone marrow was digested with Liberase TL (0.2 mg/ml) and DNase I (200 U ml-1) at 37° C. for 30 minutes. Samples were then stained with antibodies and analyzed by flow cytometry. Antibodies were purchased from Biolegend, unless otherwise stated, including fluorochrome-conjugated anti-mouse CXCR4 (clone 2B11, eBioscience), anti-CD4 (clone GK1.1), anti-CD8 (clone 53-6.7), anti-IL7 receptor (clone A7R34), anti-CD25 (clone PC61), anti-CXCR7 (clone 8F11-M16), mouse IgG2b isotype control (clone MPC-11) and rat IgG2b isotype control (clone RTK4530). Human cell lines and xenografted patient cells were labeled with anti-human CXCR4 (clone 12G5), anti-human CD45 (clone HI30) and mouse IgG2a isotype control (clone MOPC-173). Cells were analyzed using a BD LSRII or BD Fortessa (BD Biosciences). Cells were FACS purified using a MoFlo (Beckman Coulter) or BD FACS Aria II (BD Biosciences) cell sorter. FloJo software (Tree Star) was used to analyze FACS data.

Western Blot.

Whole cell lysates were prepared in RIPA buffer (50 mM Tris-HCl, 150 mM NaCl, 1% NP-40, 0.25% sodium deoxycholate, 5 mM EDTA) containing complete Mini protease and phosSTOP phosphatase inhibitors (Roche) and resolved by 8% SDS-PAGE gel electrophoresis. Proteins were transferred to nitrocellulose membranes (Bio-Rad) and probed with primary antibodies raised against c-Myc (polyclonal, Cell Signaling Technologies); 0-tubulin (polyclonal, Abcam); and β-actin (clone C4, Santa Cruz Biotechnologies). Following incubation with horseradish peroxidase (HRP)-conjugated anti-mouse or anti-rabbit secondary antibodies (Jackson laboratories), proteins were detected using the SuperSignal West Pico ECL Kit (Thermo Scientific) and exposed to film or imaged using the ChemiDoc MP system (Bio-rad).

Bone Marrow Transduction and Transplantation.

Hematopoietic stem and progenitor cells were enriched from bone marrow by magnetic selection of cells expressing c-kit (STEMCELL), and were cultured in the presence of 50 ng/mL Flt3 ligand, 50 ng/mL SCF, 10 ng/mL interleukin-7 (IL-7) and 10 ng/mL interleukin-6 (IL-6) (Peprotech). 24 and 48 hours after enrichment, c-kit+ cells were infected with concentrated retrovirus. Transduction efficiency was determined by reporter fluorescence at 96 hours. For induction of primary T-ALL, irradiated mice (two rounds of 550 rads) received 50,000 Notch1-ΔE-IRES-GFP⁺ lineage-cells, together with 500,000 unfractionated bone marrow cells for hemogenic support, by retro-orbital injection. For secondary T-ALL transplantation, recipients were sublethally irradiated (400 rads) and retroorbitally injected with 1-2×10⁶ Notch1ΔE-GFP⁺ cells from spleen or bone marrow. For niche experiments all the recipient hosts were sex-matched littermates.

Establishment of Xenografted Human T-ALL.

For human xenograft studies, NOD-SCID donor mice were used. Primary T-ALL patient samples were collected by Columbia Presbyterian Hospital with informed consent and approved and analyzed under the supervision of the Columbia University Medical Center Institutional Review Board. 1-5×10⁶ cells patient cells were transplanted into immunodeficient mouse strains via retro-orbital injection. Cells from spleen of these primary recipients were used for experiments.

In Vivo Animal Treatments.

For inhibition of CXCR4 in primary mouse T-ALL, mice were anaesthetized by isoflurane inhalation and implanted subcutaneously with osmotic pumps (model 2002; Alzet) filled with 40 mM AMD3465 (Tocris) or PBS (flow rate 0.5 ul/hour). Acute CXCR4 inhibition was achieved with 2 subcutaneous injections of 25 mg/kg AMD3465 (or PBS control) given 4 hours apart. For CXCR4 inhibition in xenografted patient T-ALL, NOD-SCID mice were implanted with osmotic pumps filled with 50 mM AMD3465 or PBS. Mice implanted with osmotic pumps were sacrificed 14 days later for analysis of tumor burden by flow cytometry.

Cell Culture and In Vitro Drug Treatments.

Primary mouse T-ALL cells isolated from spleen were maintained on OP9 stromal cells in OptiMEM+GlutaMAX (Invitrogen) supplemented with 10% FBS, 5 ng/ml IL-7, penicillin and streptomycin, and 55 μM (3-mercaptoethanol and passaged every 3-4 days onto a fresh feeder layer. OP9 cells were pre-treated with 7.5 ng/mL mitomycin C (Sigma) to prevent feeder cell division. Mouse ETP T-ALL (Treanor et al., “Interleukin-7 Receptor Mutants Initiate Early T Cell Precursor Leukemia in Murine Thymocyte Progenitors with Multipotent Potential,” J. Exp. Med. 211:701-713 (2014), which is hereby incorporated by reference in its entirety) and a T-ALL cell line generated by ENU mutagenesis carrying a Notch1 PEST domain mutation (G7084 (ins)C7085) were maintained on OP9-DLL4 feeders. AMD3100 octahydrochloride hydrate (10 ug/mL in water; Sigma), AMD3465 hexahydrobromide (50 ug/mL in water; Tocris or Cayman Chemical) or the corresponding vehicle was added to culture media at the start of the culture period.

RT-qPCR.

Total RNA was extracted from FACS purified populations using TRIzol (Invitrogen) and RNA was converted to cDNA using random hexamers and Superscript III First Strand Synthesis System (Invitrogen) according to the manufacturer's instructions. Real-time quantitative PCR (RT-qPCR) was performed using iQ SYBR Green Supermix (Bio-Rad) and a Roche LightCycler 480. Primer pairs used were: Cxcr4 forward 5′-GTAGAGCGAGTGTTGCCATG-3′ (SEQ ID NO: 6); Cxcr4 reverse 5′-TTGAAATGGACGTTTTCATCC-3′ (SEQ ID NO: 7); Hprt forward 5′-AGGTTGCAAGCTTGCTGGT-3′ (SEQ ID NO: 8); Hprt reverse 5′-TGAAGTACTCATTATAGTCAAGGGCA-3′ (SEQ ID NO: 9); Cxcl12 forward 5′-TGCATCAGTGACGGTAAACCA-3′ (SEQ ID NO: 10); Cxcl12 reverse 5′-CACAGTTTGGAGTGTTGAGGAT-3′ (SEQ ID NO: 11); Cdca7 forward 5′-CGCGCGGCAAAAGGCTCTCAAA-3′ (SEQ ID NO: 12); Cdca7 reverse 5′-CCGGCACTGACTTCGGGTCC-3′ (SEQ ID NO: 13); Pa2g4 forward 5′-TGTCGGGCGAAGACGAGCAG-3′ (SEQ ID NO: 14); Pa2g4 reverse 5′-GATCGAAGCACCCGGTTGGCG-3′ (SEQ ID NO: 15); Cks2 forward 5′-GACGTAGAGCCCCTTGCGCC-3′ (SEQ ID NO: 16); Cks2 reverse 5′-GACATGCCGGTACTCGTAGTGCT-3′ (SEQ ID NO: 17); Mad2L1 forward 5′-TGGTGGAACAGCTAAAAGAGTGGC-3′ (SEQ ID NO: 18); Mad2L1 reverse 5′-TCATCCTGTATGGCTTTCTGGGACT-3′ (SEQ ID NO: 19); Bub1b forward 5′-GAGCGCCCAGCAGACAGTCA-3′ (SEQ ID NO: 20); Bub1b reverse 5′-TGGTCAACAGCTCGGCTTCCC-3′ (SEQ ID NO: 21); Myc forward 5′-CCACCAGCAGCGACTCTGAAGAA-3′ (SEQ ID NO: 22); Myc reverse 5′-GGGTGCGGCGTAGTTGTGCT-3′ (SEQ ID NO: 23); Actb forward 5′-GGCTGTATTCCCCTCCATCG-3′ (SEQ ID NO: 24); Actb reverse 5′-CCAGTTGGTAACAATGCCATGT-3′ (SEQ ID NO: 25).

RNA Extraction and Library Construction for Next Generation Sequencing.

Total RNA was extracted from samples using RNeasy Plus Mini Kit (Life Technologies). Samples were then subject to PolyA selection using oligo-dT beads (Life Technologies) according to the manufacturer's instructions. The resulting RNA samples were then used as input for library construction using the dUTP method as described (Parkhomchuk et al, “Transcriptome Analysis by Strand-specific Sequencing of Complementary DNA,” Nucl. Acids Res. 37:e123, which is hereby incorporated by reference in its entirety). RNA libraries were then sequenced on the Illumina HiSeq 2000 or 2500 using 50 bp single-end reads. All RNA-Seq data was aligned to mm9 using TopHat v1.4 (Trapnell et al, “TopHat: Discovering Splice Junctions with RNA-seq,” Bioinformatics 25:1105-1111 (2009), which is hereby incorporated by reference in its entirety) with default parameters. Cuffdiff v1.3 (Trapnell et al, “TopHat: Discovering Splice Junctions with RNA-seq,” Bioinformatics 25:1105-1111 (2009), which is hereby incorporated by reference in its entirety) was used for all differential expression (DE) analyses with the RefSeq annotation. In all DE tests, a gene was considered significant if the qvalue was less than 0.05 (Cuffdiff default).

Histopathology and Immunofluorescent Labeling.

Tissues were dissected from euthanized animals and fixed overnight in 4% paraformaldehyde (PFA) at 4° C. For H&E sectioning the organs were dehydrated in 70% ethanol and embedded in paraffin. Bones were decalcified in 14% EDTA for 48 hours prior to dehydration and embedding. 5 μm paraffin sections were stained with hematoxylin and eosin for bright field microscopy. For analysis by immunofluorescence, fixed tissues were placed in 30% sucrose solution for 24 hours and subsequently frozen in OCT. Bones were decalcified in 0.5 M EDTA solution (Fisher Scientific) for 6 days at 4° C., placed in 30% sucrose solution for 24 hours at 4° C., and frozen in OCT. 6-8 mm cryostat sections were stained with anti-GFP antibody at 4° C. overnight. Prior to staining, bone marrow sections were dried overnight at RT in the dark. The sections were stained with DAPI for 5 min and mounted with Fluromount-G (Southern Biotech). Images were taken using Leica TCS SP5 II Confocal Microscope and analyzed on Leica Microsystems imaging software.

Two-Photon Preparation Imaging.

Mice were anesthetized using isoflurane and secured on a warming imaging plate in a supine position. The medial or soleus region of the tibial bone was surgically exposed removing soft tissue. The bone was carefully thinned to approximately 200 micron thickness using a microdrill. The leg was imbedded in agarose to stabilize the area and create an immersion well for the microscope objective. Images were collected on an Olympus FV-1000MPE upright laser scanning microscope with 25×1.05NA water immersion objective using a Spectra-physics DeepSee-MaiTai Ti:sapphire pulsed laser for excitation, with emission filters for the detection of GFP and rhodamine (Tomato or dsRed fluorescent proteins) and qdot 705 (Invitrogen). Images were analyzed on Volocity 6.3 (Improvision).

Cell Co-Localization.

High-resolution image z-stacks were taken by intravital bone marrow imaging, using similar PMT and laser power settings for all conditions and were analyzed for cell-cell contacts using Volocity 6.3 (Improvision). Tumor (GFP⁺) and stromal (tdTomato⁺ or dsRed⁺) cell populations were identified as objects using custom written protocol scripts. To separate high-intensity red fluorescence signal (from tdTomato- or dsRed-positive cells) bleeding into weaker GFP⁺ signal, ratios of channels were used to identify GFP⁺ cells, which excluded pixels that were GFP⁺ tdTomato⁺, by design. Once bona fide GFP⁺ objects were identified, object volumes were dilated once, automatically, and any GFP⁺ objects that touched Tomato/dsRed cells were enumerated as a frequency of total GFP⁺ cells in the field. The same protocol was reused for all conditions. Each automatic analysis was confirmed by eye to ensure proper cell identification. Data points reflect average values of all cells in a unique field, with ˜10-100 cells per field of bone marrow. Plots were pooled from 3 or more mice from independent experiments. Unpaired non-parametric t-tests were used to compare means from different conditions.

Statistical Analysis.

Statistical analysis (excluding RNA-Seq experiments) was conducted using the PRISM program (GraphPad). Two unpaired groups were compared using the Mann-Whitney test. More than two groups were compared using the Kruskal-Wallis test. Significance was defined as *p<0.05, **p<0.01, ***p<0.001, ****p<0.0001.

### Example 1

### Visualization of CXCL12-Rich T-ALL Niches in the Bone Marrow

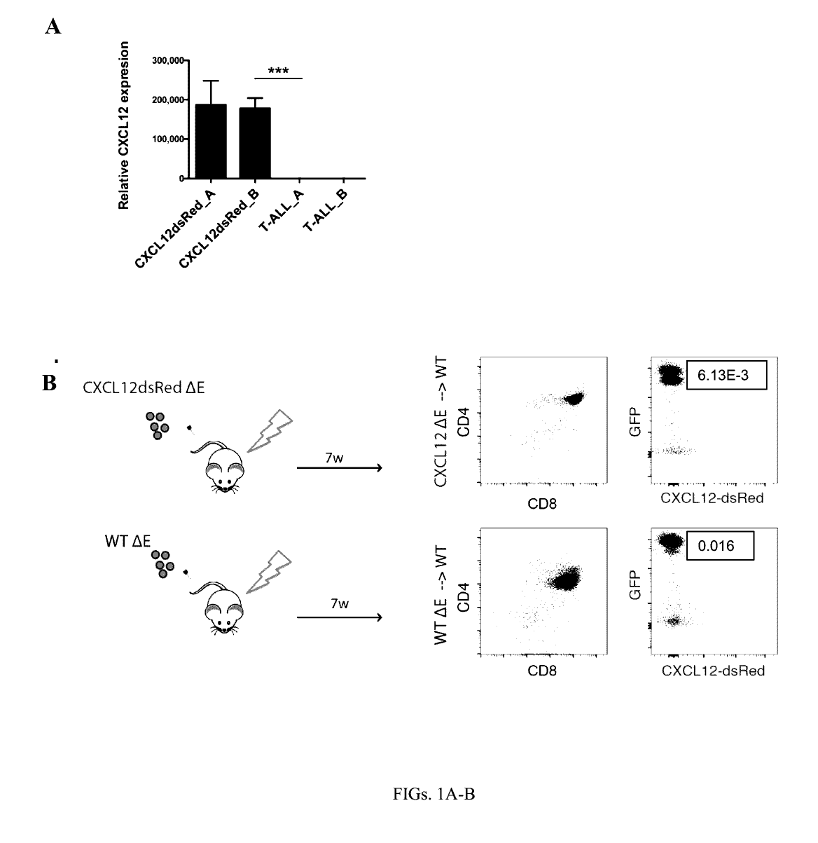

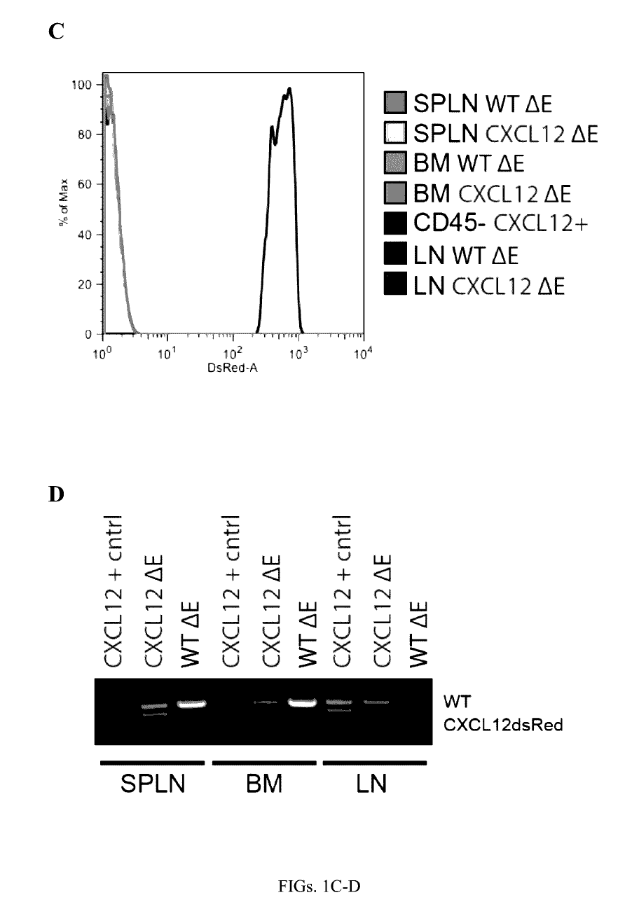

It was hypothesized that CXCL12 produced by the bone marrow stroma is an important exogenous factor for maintenance of leukemia, analogous to normal HSC and CLP (common lymphocyte progenitors). To model human T-ALL, T-ALL driven by mutated human NOTCH1 (Notch1-ΔE) (Aster et al., “Oncogenic Forms of NOTCH1 Lacking Either the Primary Binding Site for RBP-Jkappa or Nuclear Localization Sequences Retain the Ability to Associate with RBP-Jkappa and Activate Transcription,” J. Biol. Chem. 272:11336-11343 (1997), which is hereby incorporated by reference in its entirety) was generated. In this model, lineage negative c-Kit⁺ bone marrow progenitor cells are transduced with a retrovirus encoding Notch1-ΔE-IRES-GFP and transplanted into lethally irradiated recipient mice. The progenitor cells give rise to GFP⁺ leukemic blasts with an atypical CD4⁺CD8⁺ phenotype in peripheral blood, bone marrow, spleen, thymus, lymph nodes, liver, lung and central nervous system. It was previously suggested that leukemic cells can themselves produce niche factors, augmenting trophic effects (Colmone and Sipkins, “Beyond Angiogenesis: the Role of Endothelium in the Bone Marrow Vascular Niche,” Translational Research: the Journal of Laboratory and Clinical Medicine 151:1-9 (2008), which is hereby incorporated by reference in its entirety). qRT-PCR analysis of mouse T-ALL cells demonstrated that leukemic cells express undetectable levels of Cxcl12 (FIG. 1A). As a second test of whether T-ALL cells produce CXCL12, T-ALL was induced by transducing bone marrow stem and progenitor cells from Cxcl12-DsRed reporter mice or wild-type littermates with Notch1-ΔE-GFP retrovirus and transplanted them into irradiated syngeneic hosts (Ding and Morrison, “Haematopoietic Stem Cells and Early Lymphoid Progenitors Occupy Distinct Bone Marrow Niches,” Nature 495:231-235 (2013), which is hereby incorporated by reference in its entirety). T-ALL cells generated from Cxcl12-dsRed donors did not express detectable levels of DsRed, indicating that tumor cells were not able to produce CXCL12 (FIGS. 1B-D) in an autocrine fashion.

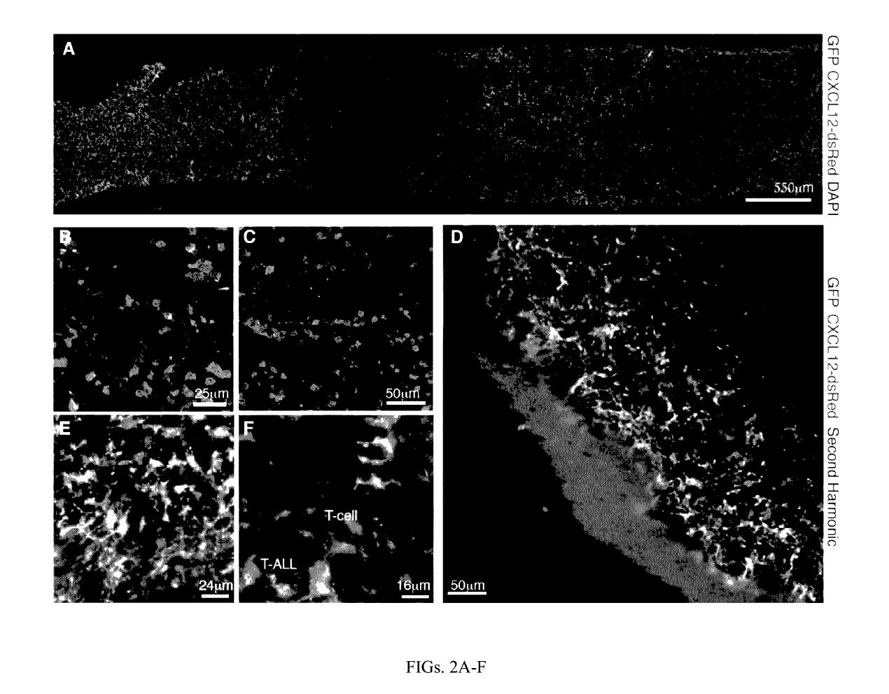

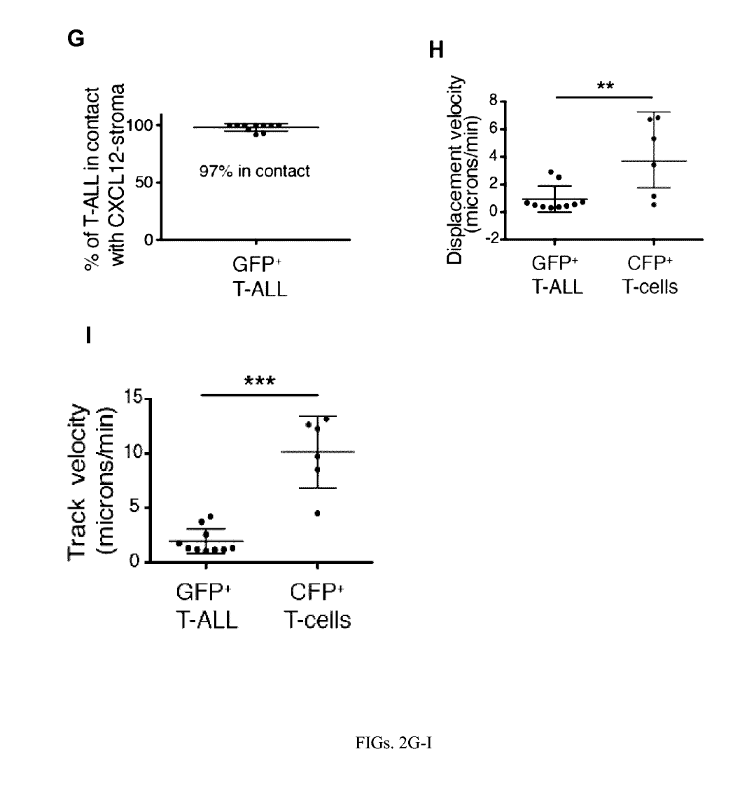

To visualize potential interactions of leukemia cells with the CXCL12-producing microenvironment, GFP⁺ T-ALL cells isolated from leukemic mice were transplanted into CXCL12-DsRed hosts. Analysis of host bone marrow sections revealed organ-wide dissemination of GFP⁺ T-ALL cells (FIG. 2A), and these cells were observed to be in direct contact with DsRed⁺ cells (FIGS. 2B-C). Time-lapse intravital imaging of an intact tibia in vivo was then performed. Mice were anesthetized, and the medial or soleus region of the tibial bone was surgically exposed and imaged. Imaging in live animals showed dissemination of leukemic cells throughout the bone marrow (FIG. 2D). High resolution analysis showed leukemic cells to be directly interacting with CXCL12-producing stroma (FIG. 2E-G). Time-lapse analysis of Cxcl12-DsRed hosts injected concomitantly with GFP⁺ T-ALL cells and CFP⁺ normal CD4⁺ T-cells revealed a dynamic microenvironment, where leukemic cells were highly immotile and stably associated with stromal cells in sharp contrast to motile CD4⁺ T-cells (FIGS. 2F, 2H-I). These studies have identified a niche for T-ALL cells and revealed unique adhesion and motility patterns of leukemic cells in the bone marrow during the early onset of disease.

### Example 2

### CXCL12 Produced by Vascular Endothelial Cells is Necessary for T-ALL Progression

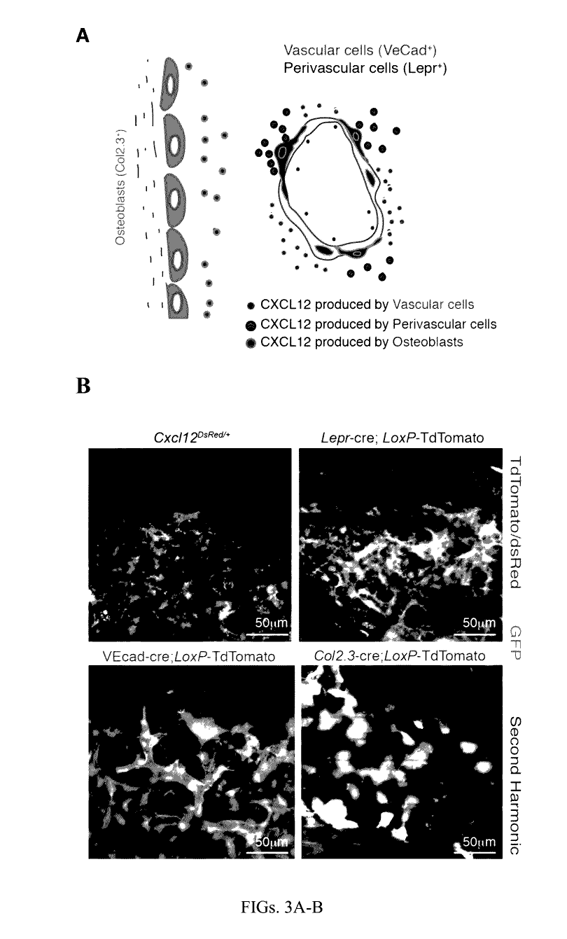

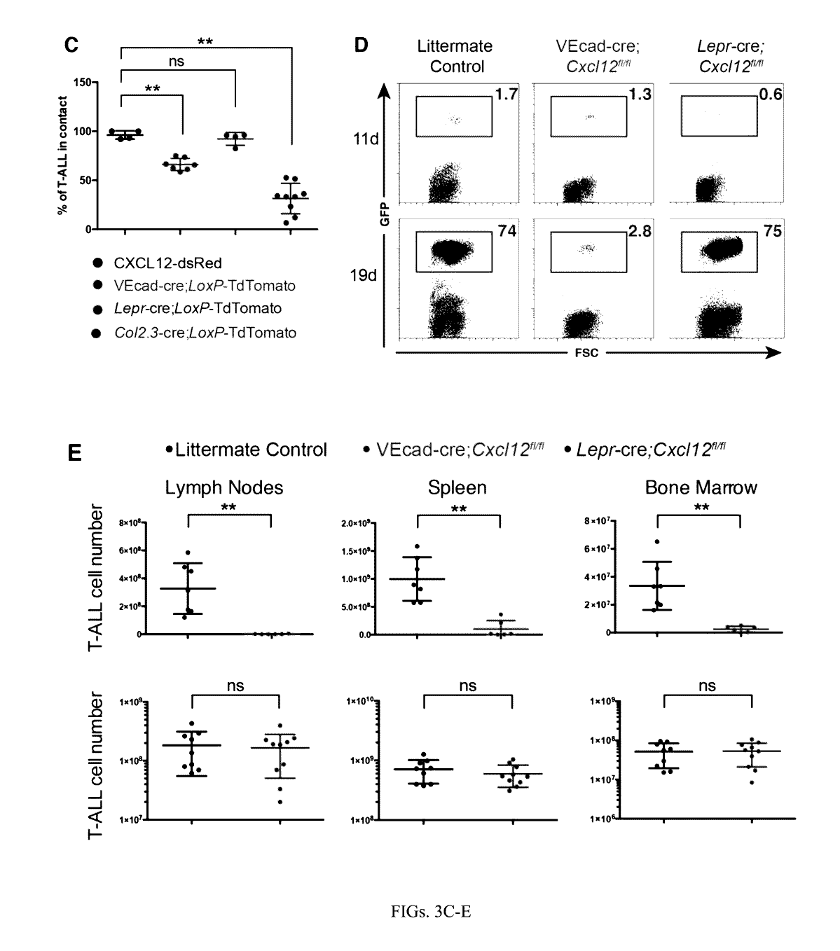

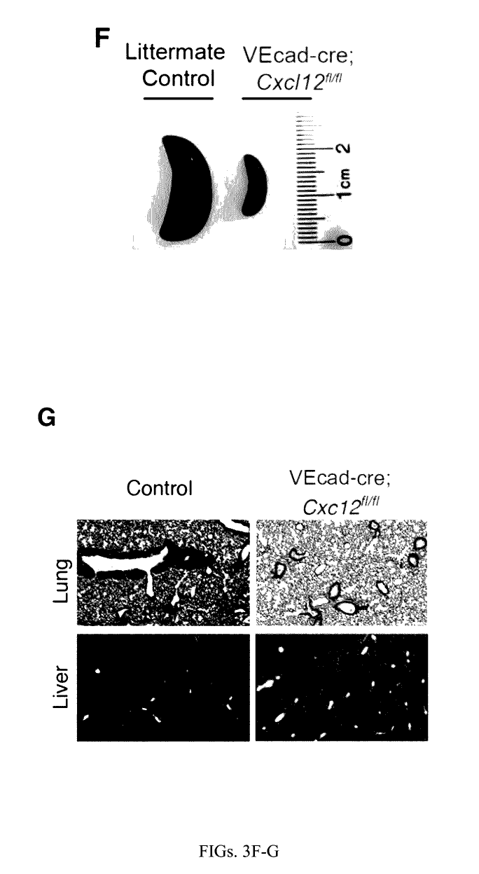

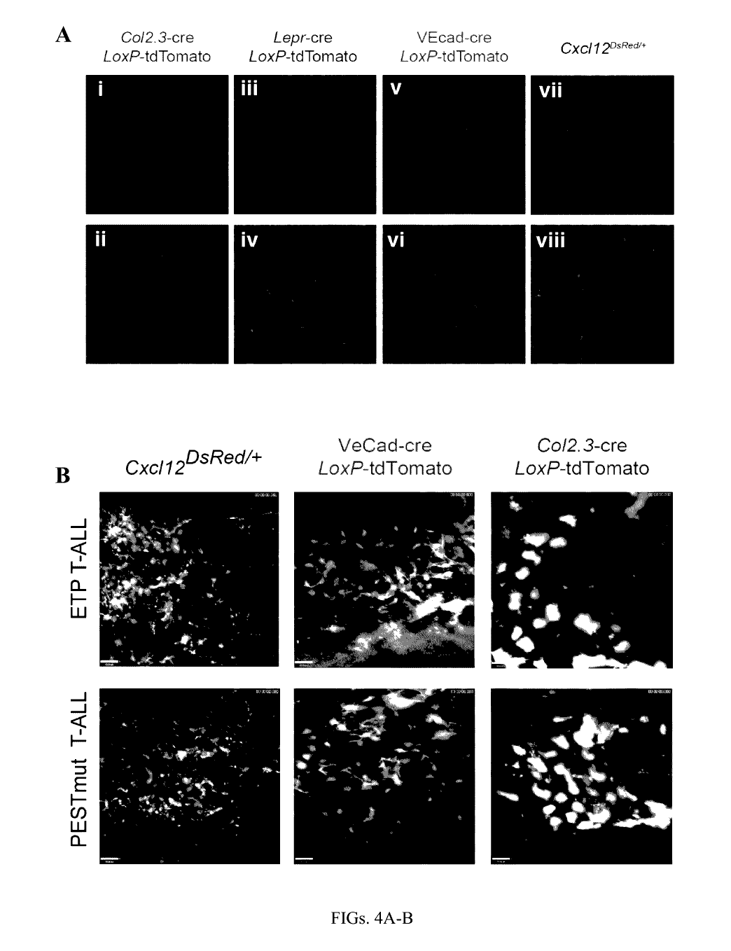

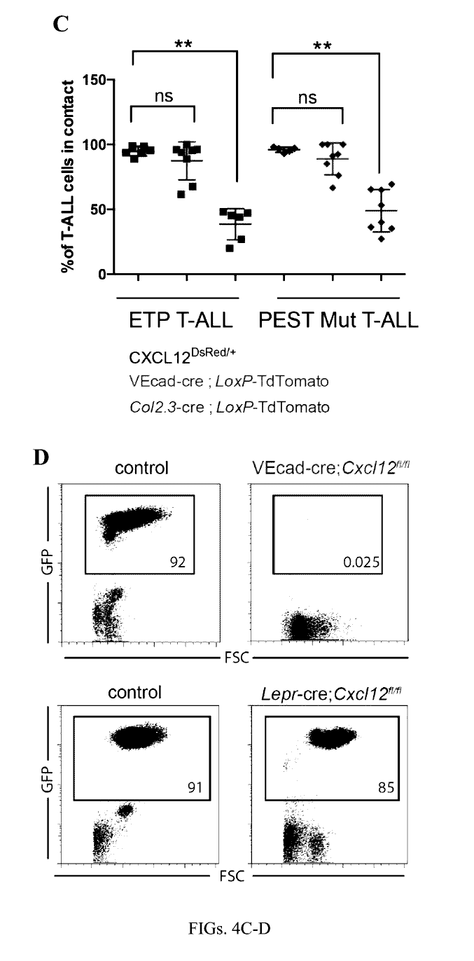

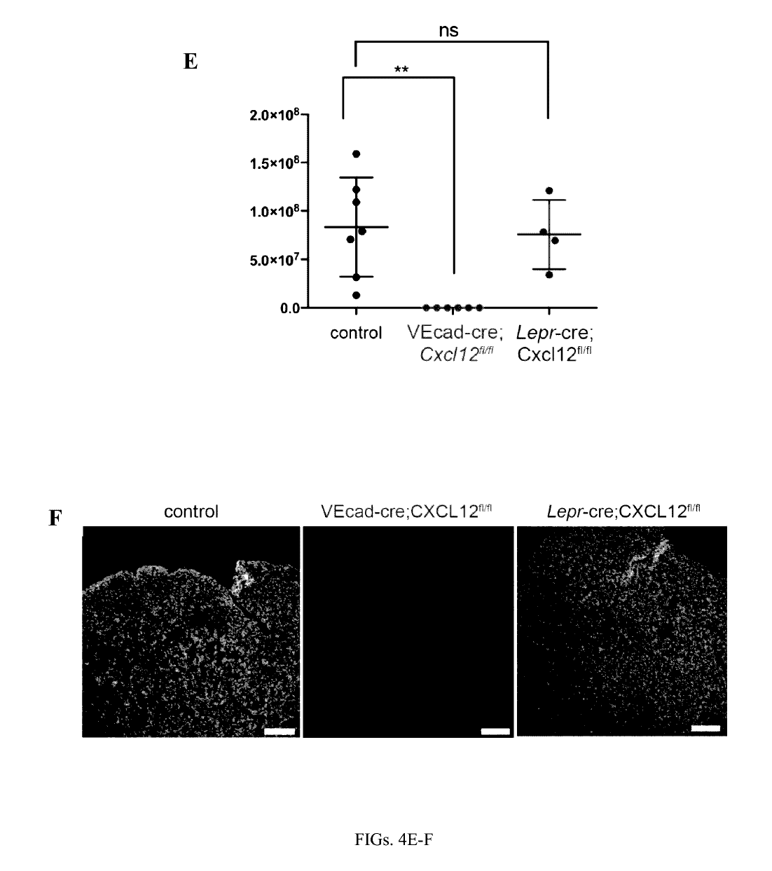

It was next tested whether CXCL12 plays an important trophic role in T-ALL. It was previously demonstrated that in the bone marrow, CXCL12 is expressed by numerous lineages including endothelial cells, perivascular cells and osteoblasts (FIG. 3A, FIG. 4A i-ii) (Ding and Morrison, “Haematopoietic Stem Cells and Early Lymphoid Progenitors Occupy Distinct Bone Marrow Niches,” Nature 495:231-235 (2013); Greenbaum et al., “CXCL12 in Early Mesenchymal Progenitors is Required for Haematopoietic Stem-cell Maintenance,” Nature 495:227-230 (2013); Sugiyama et al., “Maintenance of the Haematopoietic Stem Cell Pool by CXCL12-CXCR4 Chemokine Signaling in Bone Marrow Stromal Niches,” Immunity 25:977-988 (2006), which are hereby incorporated by reference in their entirety). To visualize these distinct niche populations in vivo, lineage-specific Cre-transgenic mice were crossed to Cre reporter mice, in which tdTomato preceded by a floxed transcriptional stop is knocked into the Rosa26 locus (LoxPtdTomato). Col2.3-cre;LoxP-tdTomato mice were used to specifically label the endosteal niche, by activating tdTomato in fetal and post-natal osteoblasts. Expression of tdTomato was seen in cells lining the bone in a pattern typical of osteoblasts (FIGS. 4A iii-iv). To label perivascular cells, Leprcre transgenic mice were crossed to LoxP-tdTomato mice (Lepr-cre;LoxP-tdTomato) (FIGS. 4A v-vi), as it was previously demonstrated that Leprin receptor is highly expressed in perivascular stromal cells (Zhou et al., “Leptin-receptor-expressing Mesenchymal Stromal Cells Represent the Main Source of Bone Formed by Adult Bone Marrow,” Cell Stem Cell 15:154-168 (2014), which is hereby incorporated by reference in its entirety). To visualize the vasculature, VEcad-cre LoxP-tdTomato mice were used. The pattern of tdTomato expression induced by VEcad-cre (FIGS. 4A vii-viii) was identical to the one observed upon intravenous injection of a VE-cadherin specific antibody.

To investigate whether leukemic cells preferentially localize with osteoblasts or the vasculature (i.e. bone marrow sinusoids) early in disease, VEcad-cre; LoxP-tdTomato, Lepr-cre;LoxP-tdTomato, and Col2.3-cre;LoxPtdTomato hosts were sublethally irradiated and injected with 1 million GFP⁺ T-ALL cells. One week later, two photon imaging analysis showed that leukemic cells preferentially associated with VE-Cad⁺ and Lepr⁺ cells, but not osteoblasts (FIG. 3B-C). These results were confirmed using two additional types of T-ALL:ETP (ll7r241-242C mutant) (Treanor et al., “Interleukin-7 Receptor Mutants Initiate Early T Cell Precursor Leukemia in Murine Thymocyte Progenitors with Multipotent Potential,” J. Exp. Med. 211:701-713 (2014), which is hereby incorporated by reference in its entirety) and an N-ethyl-N-nitrosourea (ENU)-induced mutant carrying G7084 (ins)C7085 in the PEST domain of Notch1 (FIGS. 4B-C). This was an unexpected finding, as it was previously shown that Col2.3⁺ osteoblasts constitute a niche for lymphoid progenitor cells in the bone marrow (Ding and Morrison, “Haematopoietic Stem Cells and Early Lymphoid Progenitors Occupy distinct Bone Marrow Niches,” Nature 495:231-235 (2013); Greenbaum et al., “CXCL12 in Early Mesenchymal Progenitors is Required for Haematopoietic Stem-cell Maintenance,” Nature 495:227-230 (2013), which are hereby incorporated by reference in their entirety).

To assess whether CXCL12 production by vascular endothelial cells or by closely associated perivascular cells regulates T-ALL progression, Cxcl12 was deleted in these populations by crossing Cxcl12^{fl/fl} mice to VEcad-cre (vascular) or Lepr-cre (perivascular) mice. Secondary T-ALL was then established by transplanting GFP+ T-ALL cells (10⁶) into sublethally irradiated VE-Cad-cre; Cxcl12^{fl/fl}, Lepr-cre;Cxcl12^{fl/fl}, or littermate sex-matched control hosts. Consecutive bleeds to monitor leukemia progression between day 11 and day 19 after transplantation revealed reduced expansion of T-ALL cells in mice lacking CXCL12 production specifically within the vascular compartment compared to controls (FIG. 3D). Moreover, when the mice were sacrificed on day 25 after transplantation, a significant reduction in tumor burden was observed compared to controls when CXCL12 was absent in vascular cells (FIG. 3E). Consistent with this finding, splenomegaly and thymic infiltration were not observed in VE-cad-cre; Cxcl12^{fl/fl} mice, in contrast to control animals (FIG. 3F and FIGS. 4D-F). Histo-pathological analysis also showed that T-ALL cells aggressively infiltrated non-hematopoietic tissues such as liver and lungs in control hosts, while these tissues were virtually leukemia-free in VEcad-cre;Cxcl12^{fl/fl} mice (FIG. 3G). Meanwhile, leukemia burden in Lepr-cre;Cxcl12^{fl/fl} hosts was statistically equivalent to control animals (FIGS. 3D-E). These findings demonstrate that vascular endothelial cells play a key role in leukemia progression through production of CXCL12. These findings contrast with the requirement for both perivascular and endothelial CXCL12 for hematopoietic stem cells in normal hematopoiesis.

### Example 3

### T-ALL Cells Express High Surface Levels of CXCR4

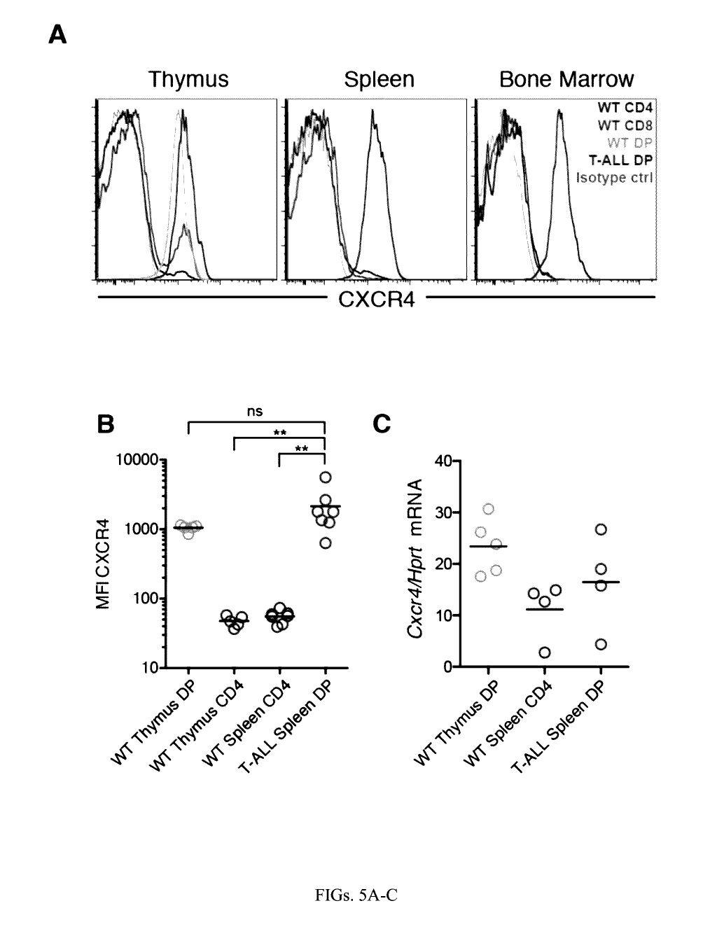

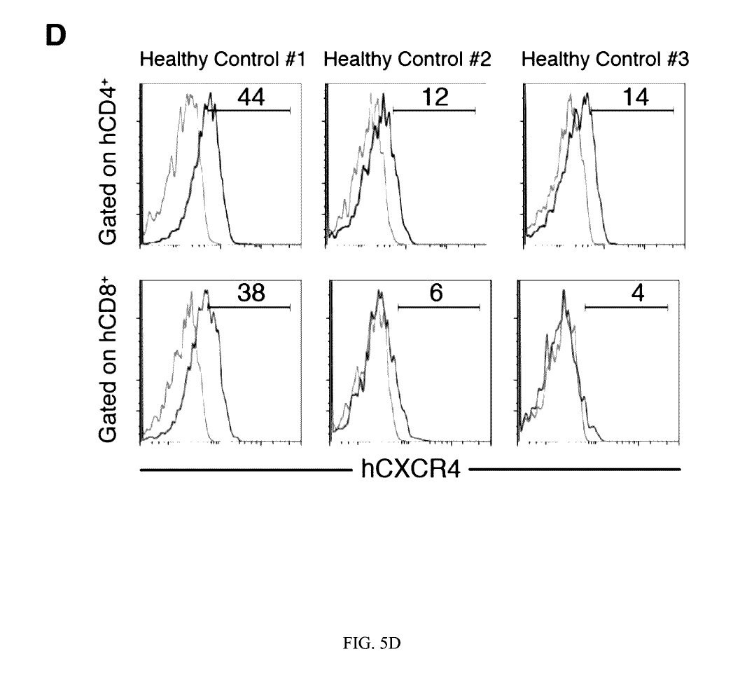

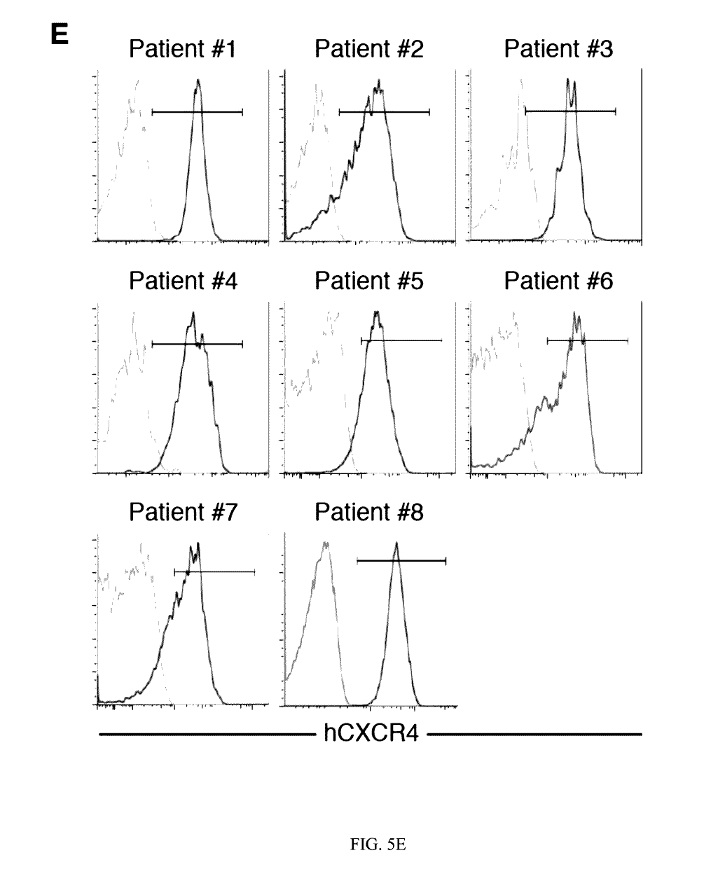

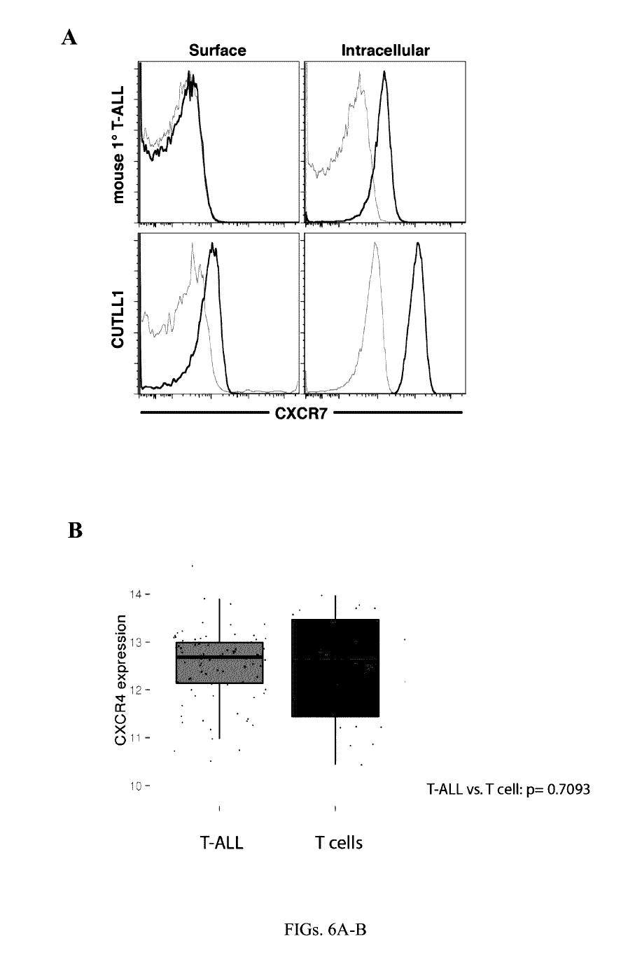

Given the importance of CXCL12 for T-ALL progression, mouse T-ALL cells were profiled for surface expression of CXCL12 receptors CXCR4 and CXCR7. It was found that primary mouse T-ALL cells express markedly high surface levels of CXCR4, but little surface CXCR7 (FIG. 5A and FIG. 6A). When CXCR4 staining was compared on the surface of primary mouse T-ALL cells in thymus, spleen and bone marrow to normal T cells from healthy mice in the corresponding tissues, it was found that the mean fluorescence intensity (MFI) of CXCR4 on T-ALL cells was 10-50 fold higher than on than mature CD4⁺ T cells and comparable to normal CD4⁺CD8⁺ (double positive [DP]) thymocytes (FIG. 5B). Notably, this pattern of elevated surface CXCR4 protein expression was not reflected in the level of Cxcr4 mRNA transcripts, as spleen T-ALL cells expressed only marginally higher levels of Cxcr4 mRNA than spleen CD4⁺ T cells and lower levels than DP thymocytes (FIG. 5C).

In agreement with these findings, global microarray analysis of T-ALL patient cohorts (Zhang et al., “The Genetic Basis of Early T-cell Precursor Acute Lymphoblastic Leukaemia,” Nature 481:157-163 (2012), which is hereby incorporated by reference in its entirety) failed to reveal significant differences in CXCR4 expression when compared to mature human T-cells (FIG. 6B). However, when CXCR4 surface expression was assessed on patient T cell leukemia cells and healthy control T cells, it was found that T-ALL cells expressed higher levels of CXCR4 than CD4⁺CD3⁺ or CD8⁺CD3⁺ T cells from the peripheral blood of healthy controls (FIGS. 5D-E). These studies demonstrate that T-ALL cells express high levels of CXCR4 (when compared with normal peripheral T cells) and suggest that these surface protein levels cannot be explained by mere transcriptional upregulation of CXCR4 mRNA.

### Example 4

### Genetic Targeting of Cxcr4 Leads to Sustained T-ALL Remission

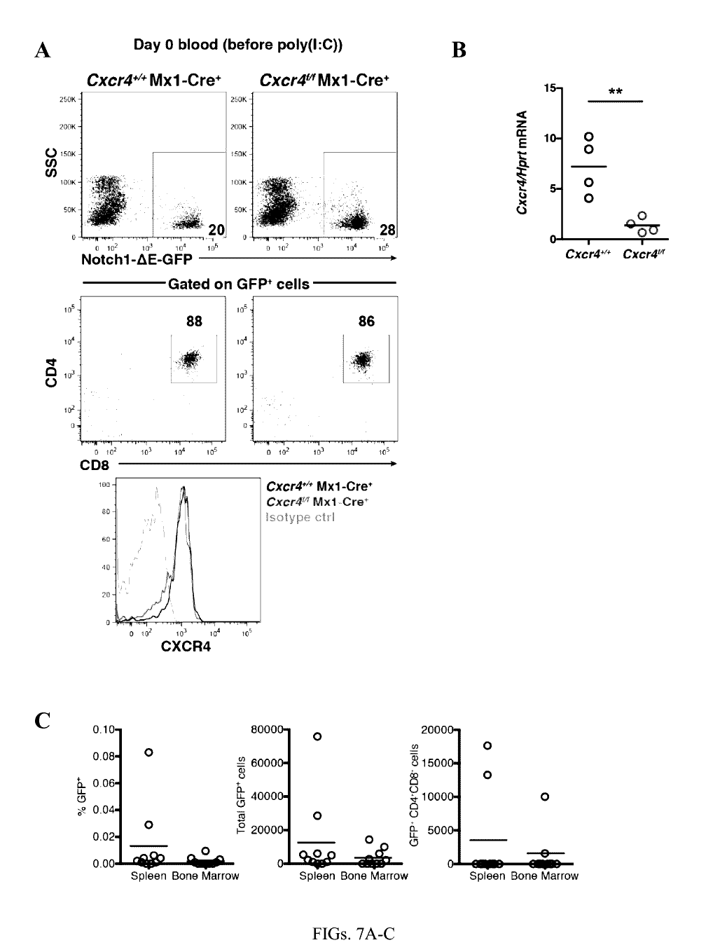

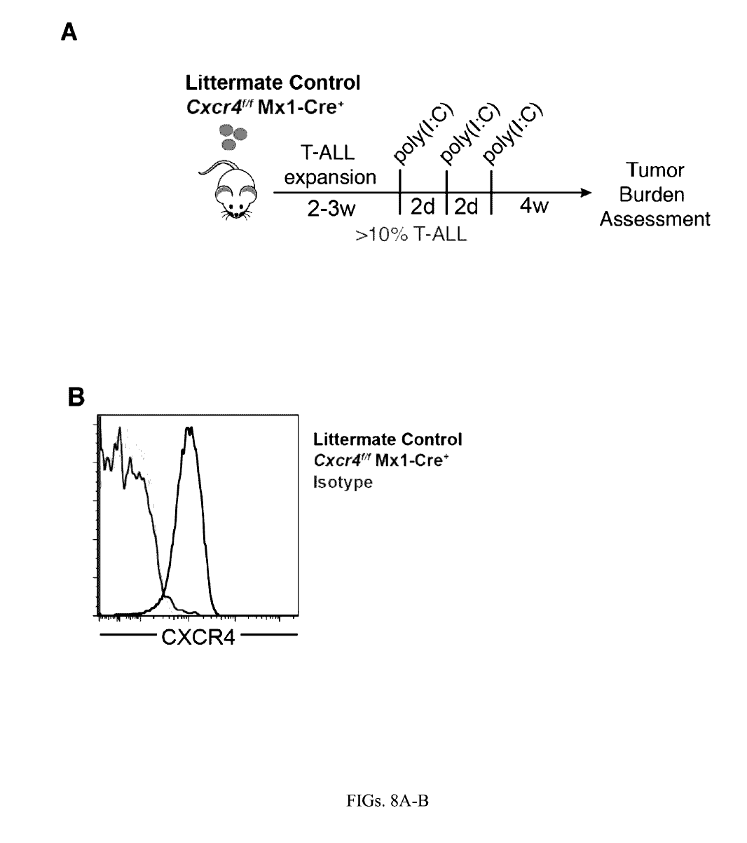

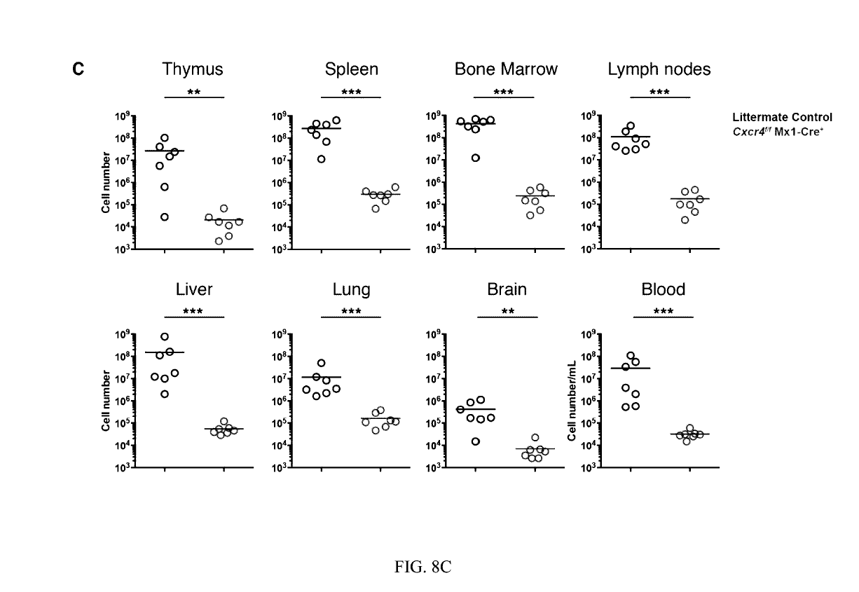

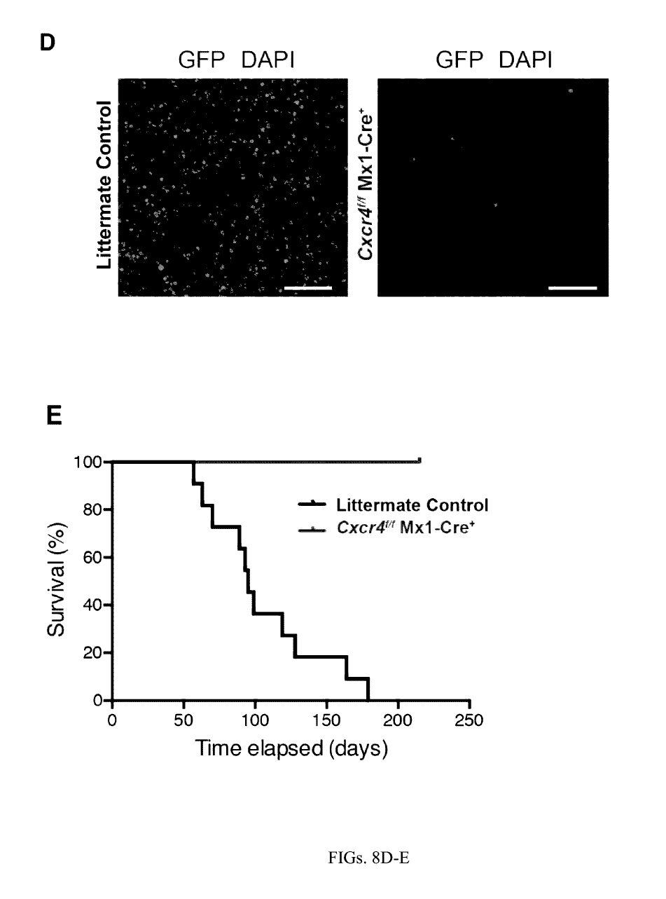

To test the requirement for CXCR4 in T-ALL progression, leukemia burden was examined following genetic ablation of Cxcr4 in vivo. Importantly, an inducible Cre recombinase was used to delete Cxcr4 after disease establishment. Bone marrow progenitor cells derived from Cxcr4^{f/f} Mx1-Cre⁺ mice or littermate control mice (Cxcr4^{+/+} Mx1-Cre⁺ or Cxcr4^{f/f}) were retrovirally transduced with Notch1-ΔE-IRES-GFP and transplanted into lethally irradiated wild-type recipients. Once GFP⁺ cells were detected in peripheral blood (>10% of lymphocytes; day 0 (FIG. 7A), both groups were treated with poly(I:C) (days 1, 3 and 5) to induce Cre transcription and Cxcr4 deletion (Kuhn et al., “Inducible Gene Targeting in Mice,” Science 269:1427-1429 (1995), which is hereby incorporated by reference in its entirety) (FIG. 8A and FIG. 7B). CXCR4 staining by flow cytometry confirmed that Cxcr4 was deleted in Cxcr4^{f/f} Mx1-Cre⁺ T-ALL cells (FIG. 8B). One month after Cxcr4 deletion, a 3-log reduction in the total number of T-ALL cells was observed, representing a loss in all tissues surveyed, including bone marrow, spleen, blood, lymph nodes, thymus, lung, liver, and brain (FIGS. 8C-D). Given the striking reduction in leukemia burden observed after Cxcr4 deletion, it was sought to determine whether survival is prolonged in mice with CXCR4-deficient T-ALL. Indeed, more than 30 weeks after deletion of Cxcr4, 100% (10/10) of mice with Cxcr4^{f/f} Mx1-Cre⁺ T-ALL cells were alive and appeared healthy, compared to 0% (11/11) of mice with Cxcr4^{+/+} Mx1-Cre⁺ T-ALL cells (FIG. 8E). Only very low numbers of GFP⁺ cells could be recovered from spleens and bone marrow of mice with CXCR4-deficient T-ALL when they were sacrificed on day 215 post-treatment with poly(I:C) (FIG. 7C). These experiments reveal the ability of Cxcr4 deletion after disease onset to lead to remission in T-ALL, and they further suggest that loss of CXCR4 signaling directly affects T-ALL initiating cell (LIC) function.

### Example 5

### CXCR4 Deletion Affects Leukemic Cell Localization and Survival

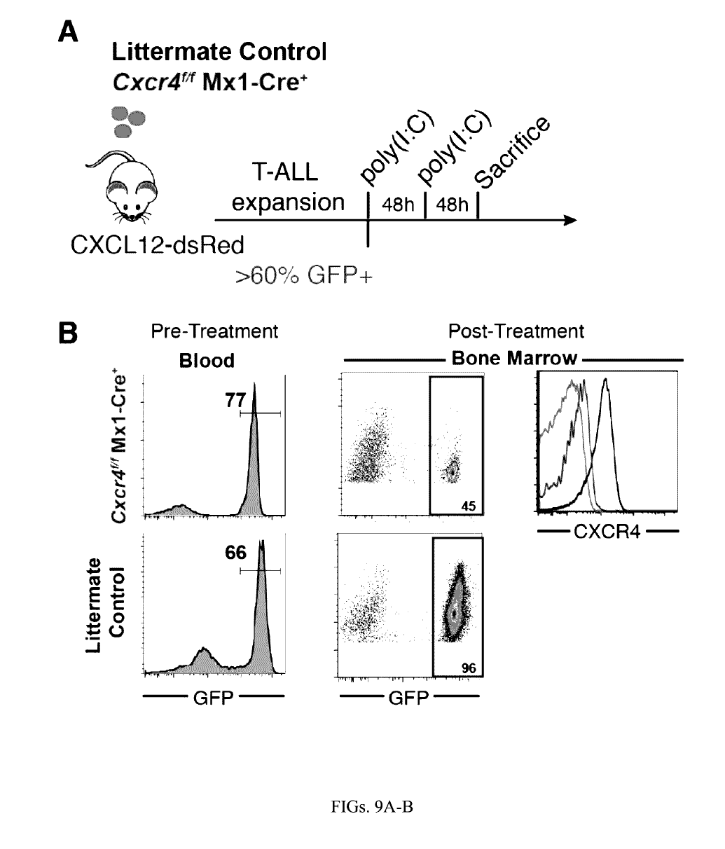

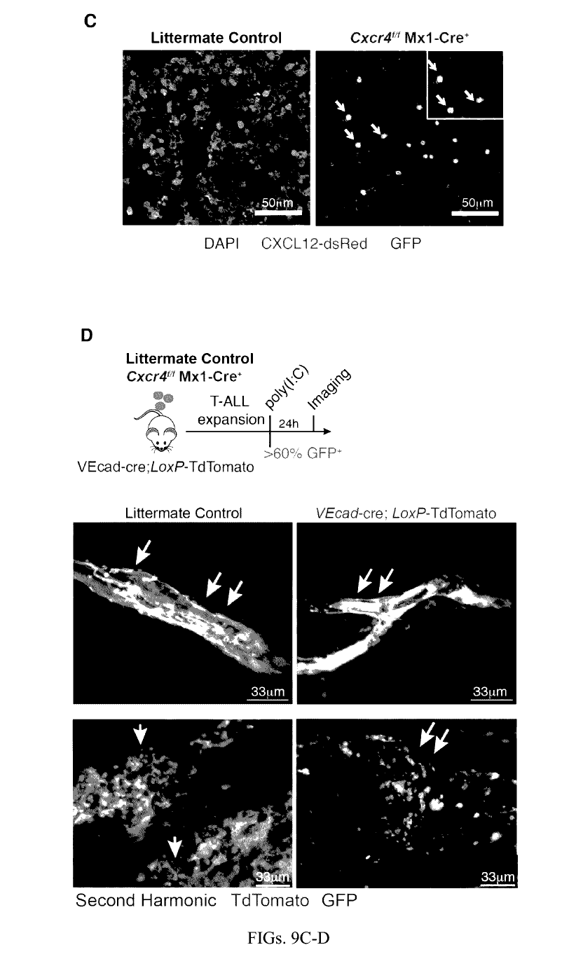

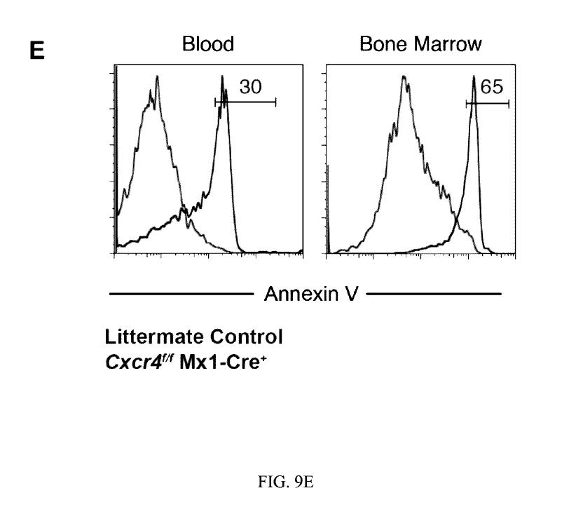

To investigate the effects of Cxcr4 deletion on T-ALL cells, secondary CXCL12-DsRed recipients were injected with equal numbers of primary Cxcr4^{f/f} Mx1-Cre⁺ or Cxcr4^{f/f} GFP⁺ T-ALL cells. When both cohorts presented 60%±10% GFP⁺ leukemic cells in the bloodstream (FIGS. 9A-B), animals were injected with two doses of poly(I:C) 48 hours apart. Both cohorts were sacrificed 48 hours after the second poly(I:C) injection and the bone marrow was examined by immunofluorescence analysis. It was found that even at this early time point, tumor cells were severely depleted from the bone marrow, and the remaining GFP⁺ cells (that had some residual surface CXCR4 expression) were localized in proximity to the CXCL12-expressing cells (FIG. 9C).

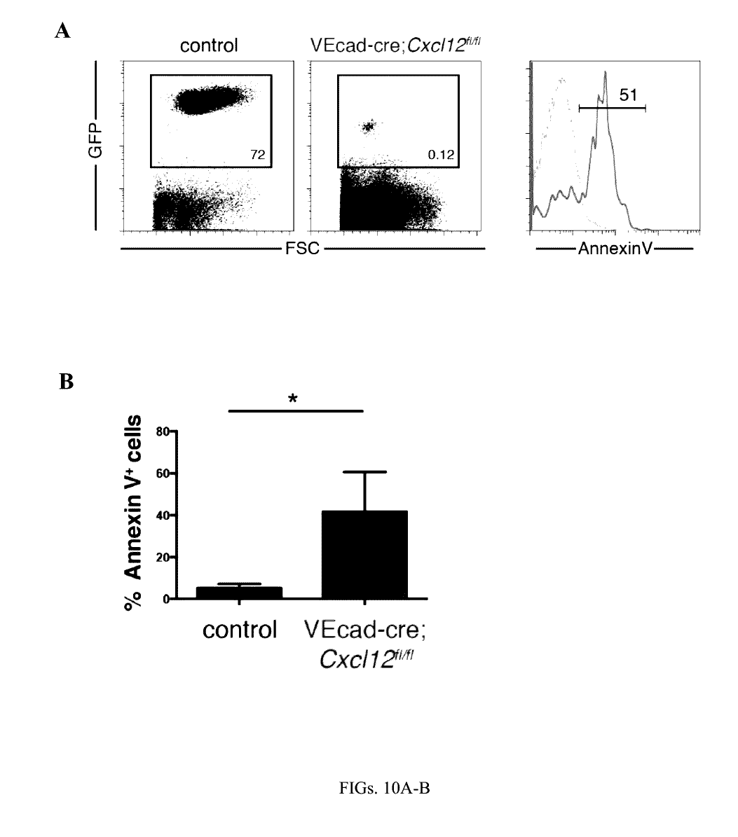

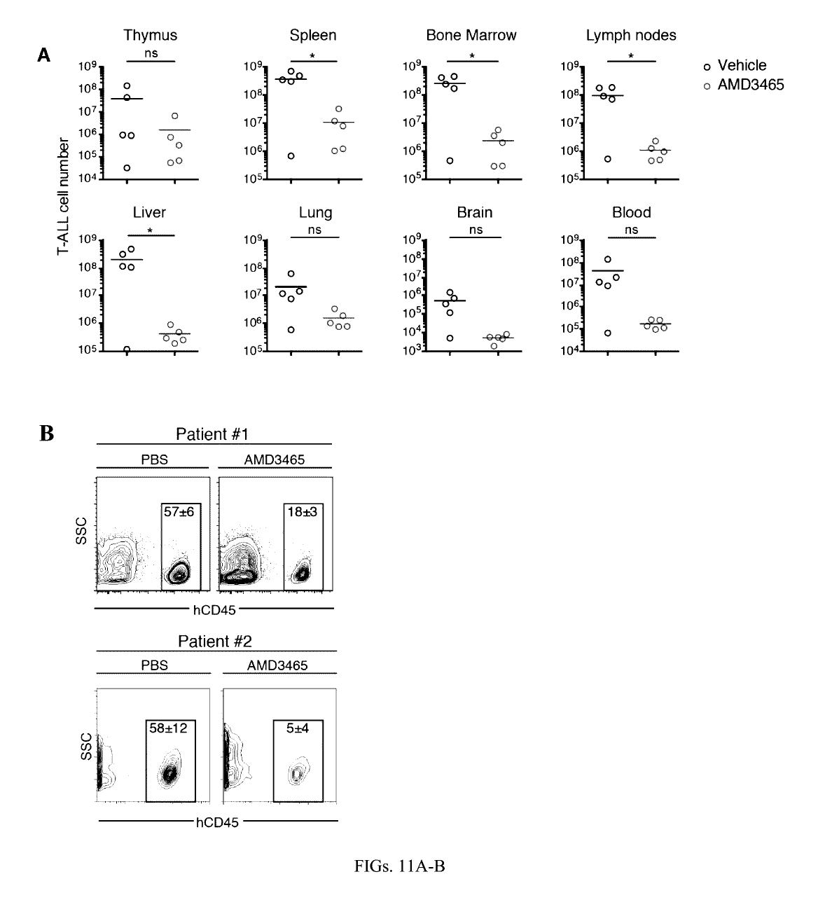

To examine the localization of CXCR4-deficient T-ALL cells in relation to vascular cells, which were found to be the key source of CXCL12, VE-Cad-cre LoxP-tdTomato hosts were transplanted with GFP⁺ Cxcr4^{f/f} Mx1-Cre⁺ or littermate control Cxcr4^{f/f} T-ALL cells. Once both cohorts presented 60%±5% GFP⁺ leukemic cells in the blood, the animals were treated with poly(I:C) and analyzed 24-72 hours later. Time-lapse intravital imaging revealed that while GFP⁺ Cxcr4^{f/f} leukemic cells were present throughout the bone marrow, Cxcr4^{f/f} Mx1-Cre⁺ cells were mainly observed within blood vessels (FIG. 9D). Moreover, poly(I:C)-injected Mx1-Cre⁺ Cxcr4^{f/f} cells showed significant levels of Annexin V staining in both the bone marrow and peripheral blood (FIG. 9E), as did wild-type T-ALL cells isolated from the spleen of VEcad-cre;Cxcl12^{f/f} hosts 6 weeks after transplantation (FIG. 10). These results suggested that CXCR4 expression and signaling influences both leukemic cell localization and survival. They also contrast effects between TALL and normal stem and progenitor cells, as in the latter populations, loss of CXCL12 leads to cell mobilization and differentiation but has not been reported to induce apoptotic death.

### Example 6

### Small Molecule CXCR4 Inhibitors Suppress Growth of Murine and Human T-ALL

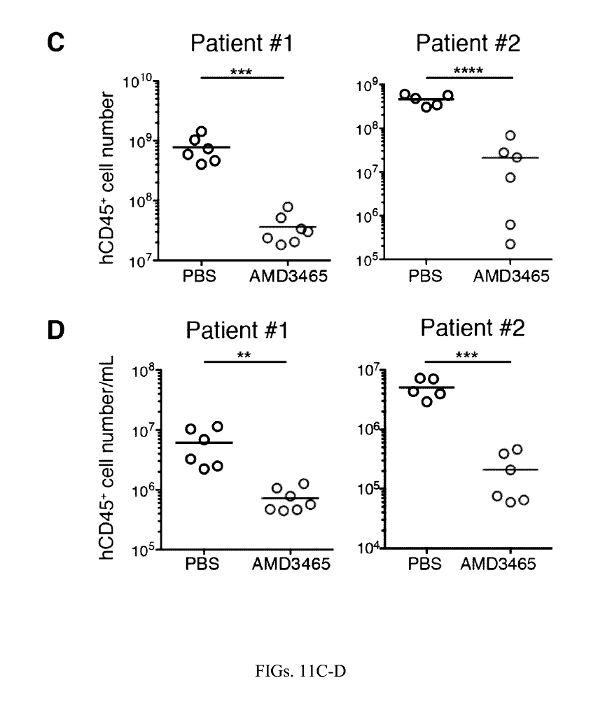

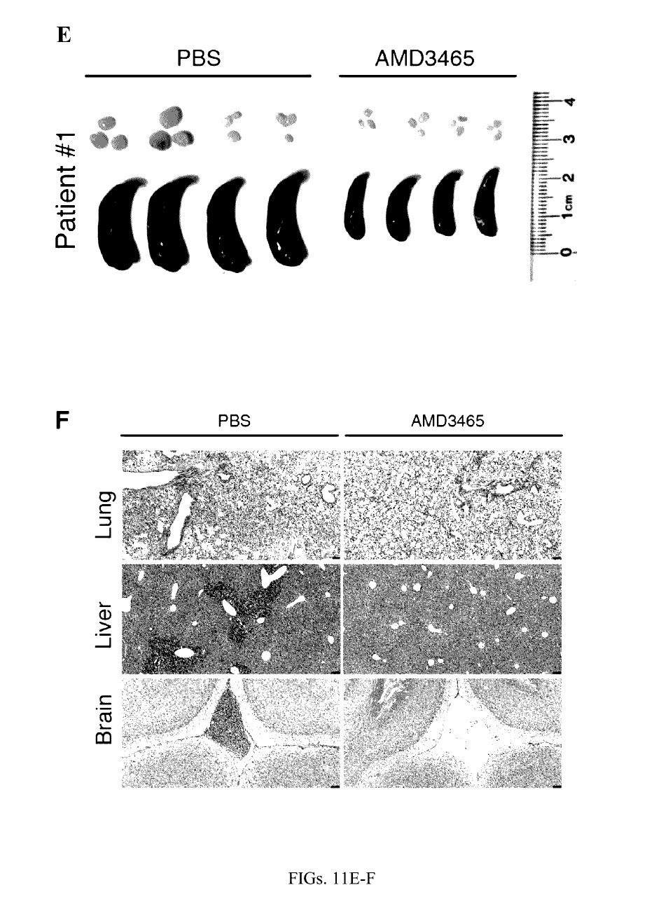

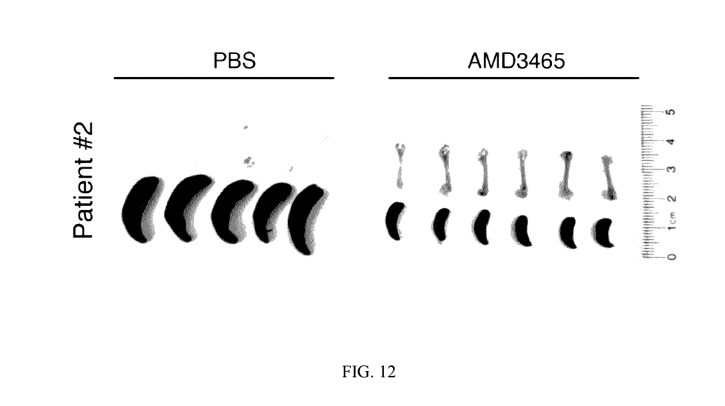

Small molecule CXCR4 antagonists have been developed as a strategy to disrupt the interaction between CXCR4 and CXCL12 and have been used in various settings, including hematopoietic stem cell mobilization (Rettig et al., “Mobilization of Haematopoietic Stem and Progenitor Cells Using Inhibitors of CXCR4 and VLA-4,” Leukemia 26:34-53 (2012), which is hereby incorporated by reference in its entirety). Therefore, it was examined whether CXCR4 antagonists can recapitulate the effects of Cxcr4 gene ablation and limit T-ALL expansion. To this end, two cohorts of lethally irradiated mice were transplanted with Notch1-ΔE-IRES-GFP⁺ bone marrow progenitor cells, and after >5% peripheral blood lymphocytes constituted GFP⁺ leukemic blasts, AMD3465 (20 nmol/hour) or PBS was administered via osmotic pump. After two weeks of treatment, mice treated with AMD3465 showed a substantial reduction in leukemia burden across all tissues surveyed (FIG. 11A). These results demonstrate the significant anti-leukemia activity of CXCR4 antagonists, even as single drugs.

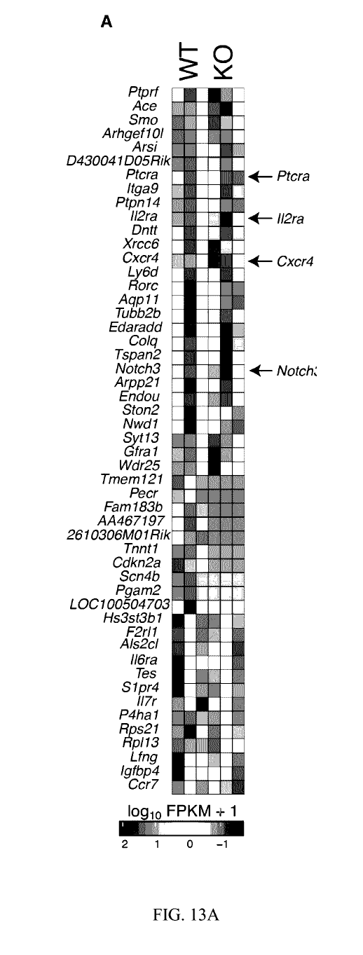

To test whether the ability of CXCR4 inhibition to suppress murine leukemia translates to human disease, the effect of AMD3465 on primary human xenografts, obtained from primary human bone marrow biopsies (corresponding to patient 1 and patient 2 in FIG. 5E), was assessed. First, the primary human leukemia cells were expanded in immune-deficient NOD/MrkBomTac-Prkdc^{scid} (NOD-SCID) hosts for 6 weeks. Subsequently, two million human T-ALL cells were transplanted into secondary NOD-SCID hosts and peripheral blood was monitored for the appearance of human CD45+ (hCD45+) leukemic cells. Once leukemia constituted >5% of white blood cells in blood, treatment began with AMD3465 or vehicle using osmotic pumps. Two weeks later, animals were sacrificed and the leukemia burden was assessed. As a result of AMD3465 treatment, the number of hCD45+ cells in blood and spleen was significantly reduced (FIGS. 11B-D). Furthermore, splenomegaly was substantially decreased in xenografted mice treated with AMD3465 compared to vehicle (PBS) (FIG. 11E and FIG. 12). Histo-pathological analysis showed that T-ALL cells in PBS treated hosts aggressively infiltrated non-hematopoietic tissues, such as brain, liver, and lungs. The same tissues in the AMD3465-treated cohort were virtually leukemia-free, highlighting the ability of AMD3465 to control primary human T-ALL progression in a xenograft model (FIG. 11F). These results provide the basis for further testing of this small molecule inhibitor in the clinical setting as a targeted T-ALL therapy.

### Example 7

### CXCR4 Controls a T-ALL-Specific Gene Expression Program and Modulates Leukemia-Initiating Activity in T-ALL

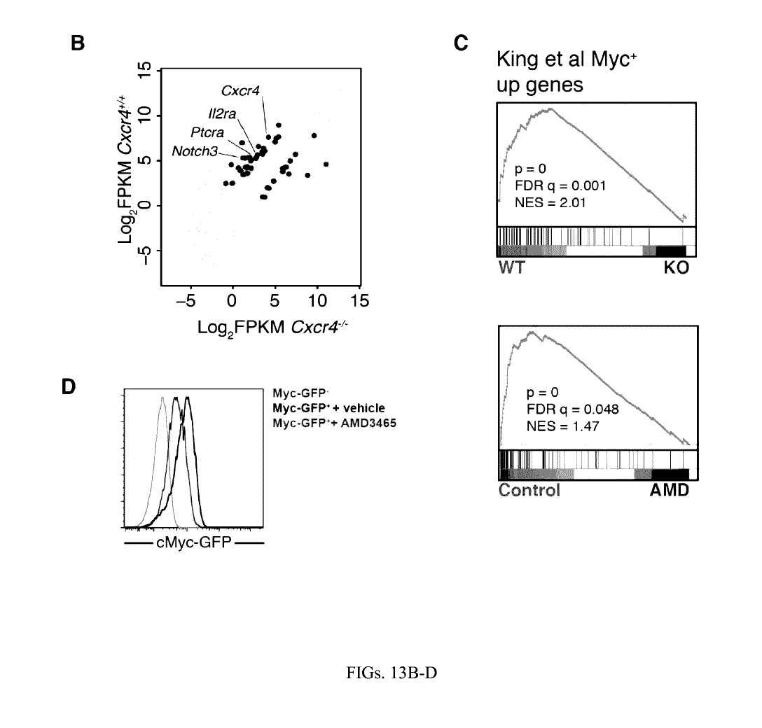

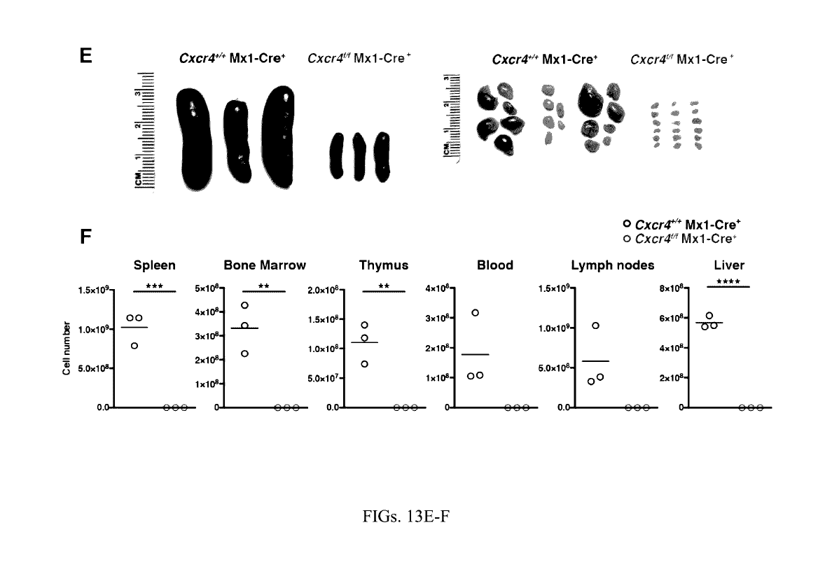

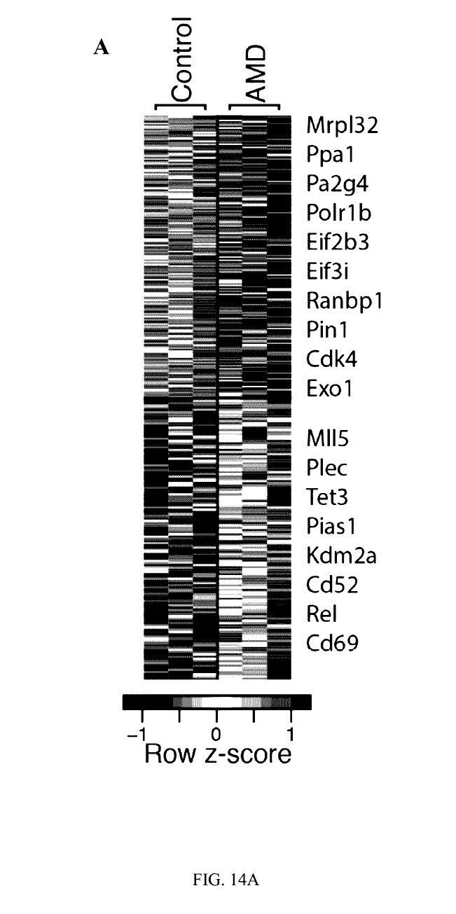

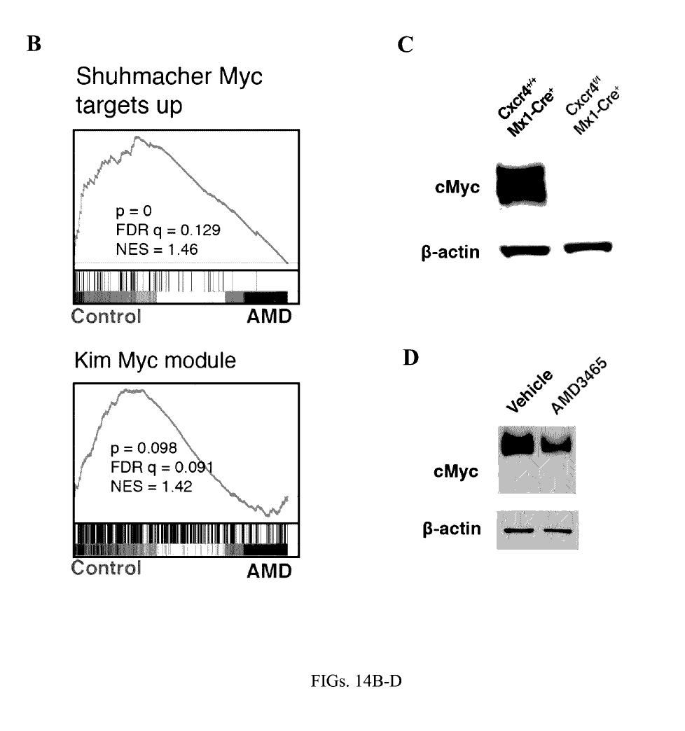

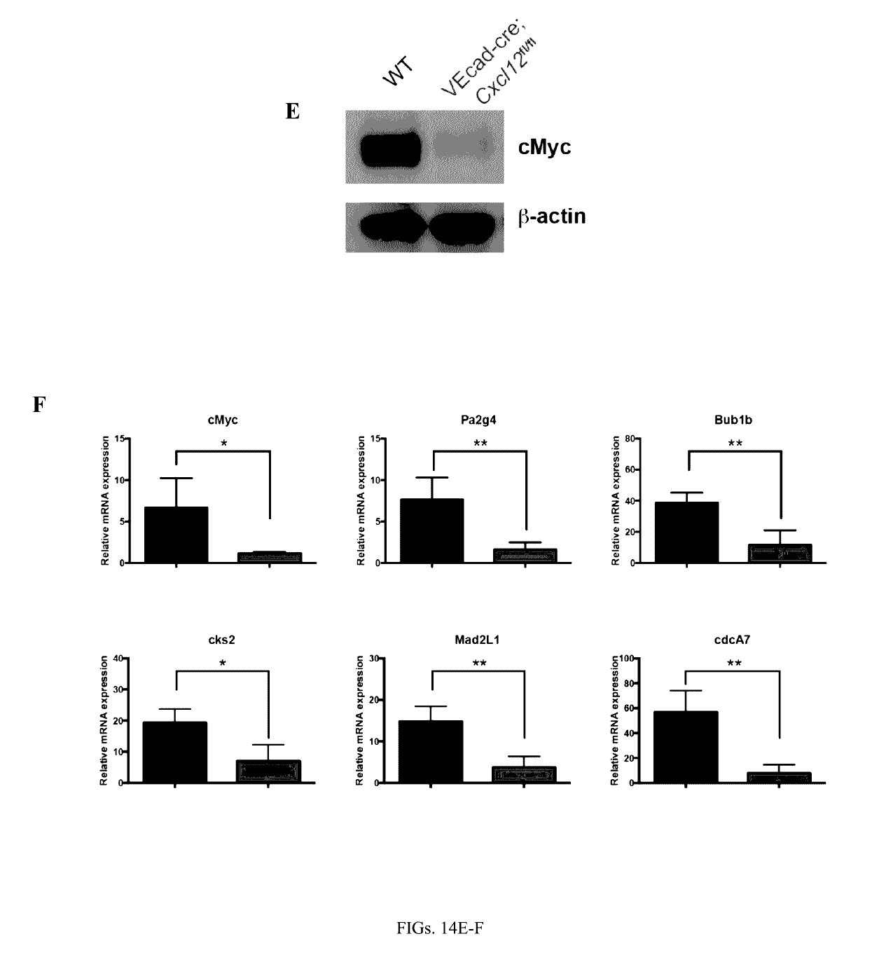

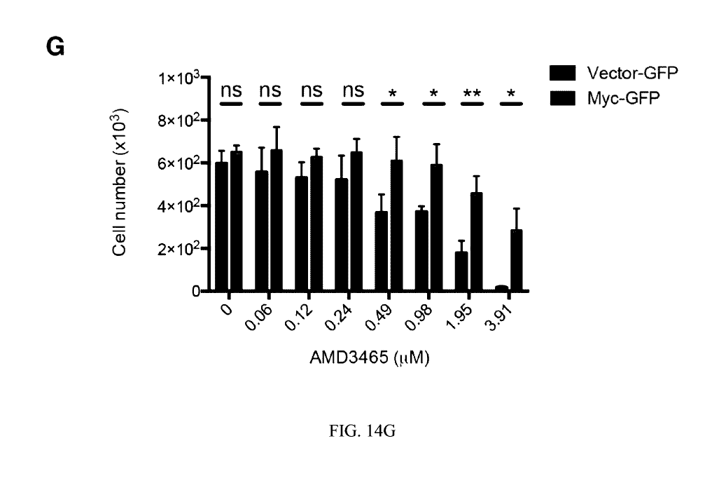

To assess the consequences of CXCR4 signaling loss on T-ALL gene transcription, high throughput transcriptome sequencing (RNA sequencing [RNA-seq]) was performed on splenic Cxcr4^{f/f} Mx1-Cre⁺ and Cxcr4^{f/f} control T-ALL cells 10 days after poly(I:C) treatment to induce Cxcr4 deletion. RNA-seq was also performed on primary splenic T-ALL cells co-cultured with OP9 cells which produce CXCL12 (Jana et al., “Thymic Development Beyond Beta-selection Requires Phosphatidylinositol 3-kinase Activation by CXCR4,” J Exp. Med. 207:247-261 (2010); Trampont et al., “CXCR4 Acts as a Costimulator During Thymic Beta-selection,” Nat. Immunol. 1:162-170 (2010), which are hereby incorporated by reference in their entirety) in the presence of the CXCR4 antagonist AMD3100 or vehicle for 4 days (FIGS. 13A-C, FIGS. 14A-B). Initial analysis of the Cxcr4-targeted data revealed changes in genes significant for early T cell development and T-ALL induction and progression such as Cdk4, Notch3, Il2ra (CD25), Ptcra, and Cdkn2α. Although altered expression of some of these genes could account for the T-ALL phenotype (i.e. Ptcra expression promotes T-ALL progression), gene set enrichment analysis (GSEA) also demonstrated a significant enrichment of genes that are down-regulated in response to AMD3100 treatment in vitro in Myc DNA binding databases (FIG. 13C and FIG. 14B) (Kim et al., “An Extended Transcriptional Network for Pluripotency of Embryonic Stem Cells,” Cell 132:1049-1061 (2008); King et al., “The Ubiquitin Ligase FBXW7 Modulates Leukemia-initiating Cell Activity by Regulating MYC Stability,” Cell 153:1552-1566 (2013); Schuhmacher et al., “The Transcriptional Program of a Human B Cell Line in Response to Myc,” Nucl. Acids Res. 29:397-406 (2001), which are hereby incorporated by reference in their entirety). Consistent with this, reduced Myc protein levels were observed in T-ALL cells purified after Cxcr4 ablation with poly(I:C) (FIG. 14C); after treatment of wild-type T-ALL cells with AMD3465 in vivo (FIG. 14D); and in wild-type T-ALL cells isolated from VEcad-cre;Cxcl12^{fl/fl} in mice (FIG. 14E), which also demonstrated reduced Myc and Myc target gene expression (FIG. 14F). Myc overexpression partially restored the proliferation of primary mouse T-ALL cells cultured in the presence of AMD3465, suggesting Myc is an important factor downstream of CXCR4 in T-ALL cells (FIG. 14G).

It was previously shown that T-ALL LIC cells are characterized by high levels of Myc protein. Furthermore, Myc deletion or silencing in established disease specifically targets LIC and leads to disease remission (King et al., “The Ubiquitin Ligase FBXW7 Modulates Leukemia-initiating Cell Activity by Regulating MYC Stability,” Cell 153:1552-1566 (2013); Roderick et al., “c-Myc Inhibition Prevents Leukemia Initiation in Mice and Impairs the Growth of Relapsed and Induction Failure Pediatric T-ALL Cells,” Blood 123:1040-1050 (2014), which are hereby incorporated by reference in their entirety). Reduced Myc protein expression was observed following AMD3465 treatment of mouse LIC cells expressing a MYC-GFP fusion allele, cultured in vitro (FIG. 13D). To assess the role of CXCR4 signaling on LIC function in vivo, the ability of CXCR4-deficient and wild-type primary T-ALL cells to establish secondary leukemia upon transfer into irradiated recipients was compared. Mice with Cxcr4^{f/f} Mx1-Cre⁺ or control Cxcr4^{+/+} Mx1-Cre⁺ primary T-ALL were treated with three doses of poly(I:C), and 3 days after the final dose, T-ALL cells were isolated from spleen and bone marrow and transferred into sublethally irradiated wild-type mice (1 million per mouse, for each genotype). Ten weeks after transplantation, recipients of poly(I:C)-treated control T-ALL cells displayed severe splenomegaly and lymphadenopathy (FIG. 13E), and fluorescence-activated cell sorting (FACS) analysis revealed that the cells had multiplied by more than three orders of magnitude in each of the tissues surveyed (FIG. 13F). In contrast, recipients of poly(I:C)-treated Cxcr4^{f/f} Mx1-Cre⁺ T-ALL cells appeared healthy, with very few or no leukemia cells detected in the same array of tissues. These data support a critical role for CXCR4 in regulating T-ALL LIC activity, in agreement with the findings showing prolonged survival (FIG. 9E) upon deletion of Cxcr4 in established T-ALL and the transcriptome profiling that connected loss of Cxcr4 expression to decreased levels of Myc protein.

### Discussion of Examples 1-7

The importance of CXCR4 in T-ALL would not have been anticipated based on its role in normal T cell maturation. T cell development is profoundly dependent, at different stages, on signaling through Notch1, pre-TCR/TCR, and IL-7R (Ciofani and Zuniga-Pflucker, “The Thymus as an Inductive Site for T Lymphopoiesis,” Ann. Rev. Cell Dev. Biol. 23:463-493 (2007); Di Santo and Rodewald, “In vivo Roles of Receptor Tyrosine Kinases and Cytokine Receptors in Early Thymocyte Development,” Curr. Opin. Immunol. 10:196-207 (1998); Radtke et al., “Deficient T Cell Fate Specification in Mice with an Induced Inactivation of Notch1,” Immunity 10:547-558 (1999), which are hereby incorporated by reference in their entirety). All three receptors and components of their signaling pathways have been implicated in T-ALL initiation and progression. By contrast, while CXCR4 signaling increases the efficiency of normal T cell development, CXCR4 is not essential for T cell maturation (Ara et al., “A Role of CXC Chemokine Ligand 12/Stromal Cell-derived Factor-1/pre-B Cell Growth Stimulating Factor and its Receptor CXCR4 in Fetal and Adult T Cell Development In Vivo,” J. Immunol. 170:4649-4655 (2003); Janas et al., “Thymic Development Beyond Beta-selection Requires Phosphatidylinositol 3-kinase Activation by CXCR4,” J Exp. Med. 207:247-261 (2010); Trampont et al., “CXCR4 Acts as a Costimulator During Thymic Beta-selection,” Nature Immunol. 11:162-170 (2010), which are hereby incorporated by reference in their entirety). However, these studies demonstrated an essential role for CXCR4 signaling in the progression of T-ALL, suggesting distinct requirements for CXCL12/CXCR4 signaling between physiology and disease.

The efficacy of CXCR4 inhibition would also not have been anticipated based on the precedent of other hematological malignancies, in which CXCR4 inhibition has been promising, but has not had dramatic effects as a single agent as the ones shown here, including rapid induction of leukemia cell death (Beider et al., “Combination of Imatinib with CXCR4 Antagonist BKT140 Overcomes the Protective Effect of Stroma and Targets CML In Vitro and In Vivo,” Mol. Cancer Thera. 13:1155-1169 (2014); Chen et al., “CXCR4 Downregulation of let-7a Drives Chemoresistance in Acute Myeloid Leukemia,” J. Clin. Invest. 123:2395-2407 (2013); Kuhne et al., “BMS-936564/MDX-1338: a Fully Human Anti-CXCR4 Antibody Induces Apoptosis In Vitro and Shows Antitumor Activity In Vivo in Hematologic Malignancies,” Clin. Cancer Res. 19:357-366 (2013); Tavor et al., “CXCR4 Regulates Migration and Development of Human Acute Myelogenous Leukemia Stem Cells in Transplanted NOD/SCID Mice,” Cancer Res. 64:2817-2824 (2004); Uy et al., “A Phase 1/2 Study of Chemosensitization with the CXCR4 Antagonist Plerixafor in Relapsed or Refractory Acute Myeloid Leukemia,” Blood 119:3917-3924 (2012); Welschinger et al., “Plerixafor (AMD3100) Induces Prolonged Mobilization of Acute Lymphoblastic Leukemia Cells and Increases the Proportion of Cycling Cells in the Blood in Mice,” Exp. Hematol. 41:293-302 (2013); Zeng et al., “Targeting the Leukemia Microenvironment by CXCR4 Inhibition Overcomes Resistance to Kinase Inhibitors and Chemotherapy in AML,” Blood 113:6215-6224 (2009), which are hereby incorporated by reference in their entirety). CXCR4 retains normal developing B-cells and myeloid cells in the bone marrow. B-cells and myeloid leukemia cells appear to share this dependence on CXCR4, as CXCR4 antagonism mobilizes these cells out of the bone marrow and into the bloodstream, depriving them of stromal support and exposing them to co-administered chemotherapeutic drugs. Loss of CXCR4 signaling may also inhibit metastasis and predispose these cells to apoptosis (Burger and Peled, “CXCR4 Antagonists: Targeting the Microenvironment in Leukemia and Other Cancers,” Leukemia 23:43-52 (2009), which is hereby incorporated by reference in its entirety). Hence, it is surprising that T-ALL appears to be more susceptible to CXCR4 antagonism than B and myeloid cancers. This may reflect a different dependence of LICs on CXCR4.

Finally, although in other tumors (e.g., AML) CXCR4 surface expression is variable, most likely reflecting the heterogeneity of the disease (Mohle et al., “The Chemokine Receptor CXCR-4 is Expressed on CD34+ Hematopoietic Progenitors and Leukemic Cells and Mediates Transendothelial Migration Induced by Stromal Cell-derived Factor-1,” Blood 91:4523-4530 (1998), which is hereby incorporated by reference in its entirety), the data herein suggest that CXCR4 expression on T-ALL is more uniform. Indeed, high surface CXCR4 has been observed on all T-ALL subtypes, including early T precursor (ETP-ALL), a more immature and high-risk disease subtype (Treanor et al., “Interleukin-7 Receptor Mutants Initiate Early T Cell Precursor Leukemia in Murine Thymocyte Progenitors With Multipotent Potential,” J. Exp. Med. 211:701-713 (2014); Zhang et al., “The Genetic Basis of Early T-cell Precursor Acute Lymphoblastic Leukaemia,” Nature 481:157-163 (2012), which are hereby incorporated by reference in their entirety). However, it cannot be excluded at this point that there are no mechanisms of resistance to CXCR4 signaling inhibition, as T-ALL can be initiated by a large spectrum of mutations, some of them altering signaling pathways (i.e., KRAS, PTEN, JAK3) or epigenetic regulators (i.e., UTX, EZH2, PHF6). It is thus conceivable that inactivation by some of these genes could lead to disease refractory to CXCR4 inhibition.

All these suggest that targeting the CXCL12-expressing microenvironment might have significant implications for the treatment of pediatric and adult T-ALL. Interestingly, recent clinical trials data using AMD3100 (Plerixafor) demonstrated that the drug is absorbed quickly and is well tolerated (McDermott et al., “A Phase 1 Clinical Trial of Long-term, Low-dose Treatment of WHIM Syndrome with the CXCR4 Antagonist Plerixafor,” Blood 123:2308-2316 (2014); Uy et al., “A Phase 1/2 Study of Chemosensitization with the CXCR4 Antagonist Plerixafor in Relapsed or Refractory Acute Myeloid Leukemia,” Blood 119:3917-3924 (2012), which are hereby incorporated by reference in their entirety). It has orphan drug status for the mobilization of HSCs and was approved by the U.S. Food and Drug Administration for this indication. While safety needs to be further validated in T-ALL patients and especially children afflicted by the disease, the results in murine and xenograft models, together with the results reported in Passaro et al., “CXCR4 is Required for Leukemia-Initiating Cell Activity on T Cell Acute Lymphoblastic Leukemia,” Cancer Cell 27:769-779 (2015), which is hereby incorporated by reference in its entirety, provide a strong foundation for the assessment of CXCR4 antagonists in clinical trials, either as a single agent or more likely in combination with established chemotherapy regimens. They also suggest the power of therapeutic targeting of the cancer microenvironment in leukemia.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating T-cell acute lymphoblastic leukemia in a subject, said method comprising:
selecting a subject having T-cell acute lymphoblastic leukemia and
administering, to the selected subject, a therapeutic agent that inhibits CXCR4-CXCL12 signaling at a dosage effective to treat the T cell acute lymphoblastic leukemia in the subject.

2. The method according to claim 1, wherein the therapeutic agent is a CXCR4 inhibitor.

3. The method according to claim 2, wherein the CXCR4 inhibitor is a small molecule selected from the group consisting of AMD3100, AMD3465, AMD070, BKT140, MSX-122, POL6326, and TG-0054

4. The method according to claim 1, wherein the therapeutic agent is a CXCL12 inhibitor.

5. The method according to claim 1 further comprising:
administering a chemotherapeutic agent to the subject in combination with said therapeutic agent.

6. The method according to claim 5, wherein the chemotherapeutic agent is selected from the group consisting of cytarabine, vincristine, prednisone, doxorubicin, daunorubicin, PEG asparaginase, methotrexate, cyclophosphamide, L-asparaginase, etoposide, and leucovorin.

7. The method according to claim 1, wherein said administering is repeated periodically.

8. The method according to claim 1, wherein T-cell acute lymphoblastic leukemia is adult T-cell acute lymphoblastic leukemia.

9. The method according to claim 1, wherein T-cell acute lymphoblastic leukemia is pediatric T-cell acute lymphoblastic leukemia.

10. The method according to claim 1, wherein T-cell acute lymphoblastic leukemia is early T-cell precursor acute lymphoblastic leukemia.

11. A method of inhibiting T-cell acute lymphoblastic leukemia cell proliferation and/or survival, said method comprising:
administering to a population of T-cell acute lymphoblastic leukemia cells an inhibitor of CXCR4-CXCL12 signaling at a dosage effective to inhibit the T-cell acute lymphoblastic leukemia cell proliferation and/or survival.

12. The method according to claim 11, wherein the inhibitor is a CXCR4 inhibitor.

13. The method according to claim 11, wherein the CXCR4 inhibitor is a small molecule selected from the group consisting of AMD3100, AMD3465, AMD070, BKT140, MSX-122, POL6326, and TG-0054

14. The method according to claim 11, wherein the therapeutic agent is a CXCL12 inhibitor.

15. The method according to claim 11, wherein said administering is carried out in vivo.

16. The method according to claim 11, wherein said administering is repeated periodically.

17. The method according to claim 11, wherein the T-cell acute lymphoblastic leukemia cells are T-cell acute lymphoblastic leukemia leukemic initiating cells.


SEQUENCE LISTING (25 sequences)

SEQ ID NO: 1 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 3 - protein, 6 aa, Homo sapiens
RFFESH

SEQ ID NO: 4 - protein, 14 aa, Artificial Sequence
  CXCR4 inhibitor
RRXCYRKKPYRXCR

SEQ ID NO: 5 - protein, 14 aa, Artificial Sequence
  CXCR4 inhibitor
RRXCYXKKPYRXCR

SEQ ID NO: 6 - DNA, 20 bp, Artificial Sequence
  primer
gtagagcgagtgttgccatg

SEQ ID NO: 7 - DNA, 21 bp, Artificial Sequence
  primer
ttgaaatggacgttttcatcc

SEQ ID NO: 8 - DNA, 19 bp, Artificial Sequence
  primer
aggttgcaagcttgctggt

SEQ ID NO: 9 - DNA, 26 bp, Artificial Sequence
  primer
tgaagtactcattatagtcaagggca

SEQ ID NO: 10 - DNA, 21 bp, Artificial Sequence
  primer
tgcatcagtgacggtaaacca

SEQ ID NO: 11 - DNA, 22 bp, Artificial Sequence
  primer
cacagtttggagtgttgaggat

SEQ ID NO: 12 - DNA, 22 bp, Artificial Sequence
  primer
cgcgcggcaaaaggctctcaaa

SEQ ID NO: 13 - DNA, 20 bp, Artificial Sequence
  primer
ccggcactgacttcgggtcc

SEQ ID NO: 14 - DNA, 20 bp, Artificial Sequence
  primer
tgtcgggcgaagacgagcag

SEQ ID NO: 15 - DNA, 21 bp, Artificial Sequence
  primer
gatcgaagcacccggttggcg

SEQ ID NO: 16 - DNA, 20 bp, Artificial Sequence
  primer
gacgtagagccccttgcgcc

SEQ ID NO: 17 - DNA, 23 bp, Artificial Sequence
  primer
gacatgccggtactcgtagtgct

SEQ ID NO: 18 - DNA, 24 bp, Artificial Sequence
  primer
tggtggaacagctaaaagagtggc

SEQ ID NO: 19 - DNA, 25 bp, Artificial Sequence
  primer
tcatcctgtatggctttctgggact

SEQ ID NO: 20 - DNA, 20 bp, Artificial Sequence
  primer
gagcgcccagcagacagtca

SEQ ID NO: 21 - DNA, 21 bp, Artificial Sequence
  primer
tggtcaacagctcggcttccc

SEQ ID NO: 22 - DNA, 23 bp, Artificial Sequence
  primer
ccaccagcagcgactctgaagaa

SEQ ID NO: 23 - DNA, 20 bp, Artificial Sequence
  primer
gggtgcggcgtagttgtgct

SEQ ID NO: 24 - DNA, 20 bp, Artificial Sequence
  primer
ggctgtattcccctccatcg

SEQ ID NO: 25 - DNA, 22 bp, Artificial Sequence
  primer
ccagttggtaacaatgccatgt